(19) United States
(12) Patent Application Publication
(10) Pub. No.: US 2020/0306222 A1
(43) Pub. Date: Oct. 1, 2020

(54) Dosing Regimen of Siponimod

(71) Applicant: NOVARTIS AG, Basel (CH)

(72) Inventors: Jang-Ho Cha, Arlington, MA (US); Frank Dahlke, Basel (CH); Anne Gardin, Folgensbourg (FR); Eric Legangneux, Levallois-Perret (FR); Carl Joseph Malanga, III, Cambridge, MA (US); Kasra Shakeri-Nejad, Wiesental (DE); Erik Wallstrom, Basel (CH); Christian Wolf, Uccle (BE)

(21) Appl. No.: 16/651,954

(22) PCT Filed: Sep. 27, 2018
(86) PCT No.: PCT/IB2018/057493

§ 371 Date: Mar. 27, 2020

Related U.S. Application Data
(60) Provisional application No. 62/565,269, filed on Sep. 29, 2017.

(51) Int. Cl. A61K 31/397; A61K 9/00; A61P 9/10; A61K 47/40

(52) U.S. Cl. CPC A61K 31/397; A61K 47/40; A61P 9/10; A61K 9/0019

(57) ABSTRACT

The present disclosure relates to a novel dosing regimen for the administration of siponimod or pharmaceutically acceptable co-crystals or salts thereof, in the treatment of stroke, in particular intracerebral hemorrhage (ICH).

### FIELD OF THE INVENTION

The present disclosure relates to a safe and efficacious method of treatment of stroke with siponimod, or pharmaceutically acceptable salts, co-crystals, hydrates, solvates, polymorphs and/or mixtures thereof. In particular it relates to a method of treatment of hemorrhagic stroke and more in particular to the treatment of intracerebral hemorrhage (ICH).

The present disclosure further relates to a dosing regimen for the administration of siponimod, or pharmaceutically acceptable salts, co-crystals, hydrates, solvates, polymorphs and/or mixtures thereof, in the treatment of stroke, in particular in the treatment of ICH.

### BACKGROUND OF THE INVENTION

Stroke remains one of the leading causes of adult death and disability worldwide. Stroke is a medical condition in which poor blood flow to the brain results in cell death, i.e. the blood supply to the brain is interrupted or reduced. There are two main types of stroke: ischemic, e.g. acute ischemic stroke, due to lack of blood flow caused by a blocked artery, and hemorrhagic, e.g. intracerebral haemorrhage (ICH), due to bleeding. They result in part of the brain not functioning properly.

Intracerebral hemorrhage (ICH)), which is basically bleeding within the brain itself (when an artery in the brain bursts, flooding the surrounding tissue with blood), due to either intraparenchymal hemorrhage (bleeding within the brain tissue) or intraventricular hemorrhage (bleeding within the brain's ventricular system).

Intracerebral hemorrhage (ICH) occurs when diseased blood vessels rupture, causing hemorrhage into brain tissue. The most common risk factors associated with ICH are hypertension, smoking, and diabetes mellitus. ICH is frequently associated with hypertensive cerebral microangiopathy in the basal ganglia and brain stem, with cerebral amyloid angiopathy in the cortical arteriolar and venular microvessels of elderly patients, and with the use of oral anticoagulants.

Intracerebral haemorrhage (ICH) is the second most frequently observed subtype of stroke after ischemic stroke. ICH constitutes 10-15% of all strokes, and is a common cause of morbidity and mortality (Qureshi et al. 2001). Although ICH accounts for a minority of all cases of stroke, it results in 50% of stroke deaths, contributing disproportionately to stroke morbidity and mortality (Qureshi et al. 2001, Asuzu et al. 2016). Clinical outcomes of the condition are poor: only 20% of patients achieve functional independence at 6 months after ICH (van Asch, C. J. et al., 2010). Patients who survive typically have major neurological impairments.

ICH causes primary brain injury through direct mechanical effects of the hemorrhage and also triggers a complex cascade of events in the affected tissue, including inflammatory processes and edema formation (Urday et al. 2015). Said events contribute to the formation of perihematomal edema (PHE), a secondary brain injury, which evolves over hours to days and contributes to neurological deterioration after ICH.

Pathophysiology of the PHE involves thrombin-induced activation of resident microglia leading to secretion of cytokines, including tumor necrosis factor (TNF) and interleukin IL-1β. These pro-inflammatory cytokines induce the breakdown of the blood-brain barrier, which consequently leads to the influx of peripheral inflammatory cells to the perihematomal region. PHE increases in volume and thus enhances the initial damage triggered by bleeding. Therefore PHE may itself augment the mass effect of the haemorrhage. It is also believed that PHE results from the same processes that cause other types of secondary brain injury, and the volume of PHE reflects the activity of pathological mediators that underlie these processes, including cytokines, complement proteins and matrix metalloproteinases (MMPs). This overlap makes PHE a valuable marker of secondary injury that could provide a useful surrogate end point for experimental and clinical studies of novel therapeutic agents to prevent secondary injury following ICH.

Neuroinflammation after ICH involves the early activation of resident microglia, release of pro-inflammatory mediators that likely contribute to the pathophysiology of secondary brain damage. Lymphocytes were found in human cerebrospinal fluid early, starting at 6 hours post ICH, and were also detected in perihematomal edema (PHE) in ICH patients. It was found that CD4+T cells are the predominating lymphocyte population in mice on day 1. Along with other T cell populations, proinflammatory and immunosuppressive regulatory T cells infiltrate the hemorrhagic brain (Mrasco and Veltkamp 2014). Despite this information on the infiltration pattern of lymphocytes in experimental ICH, less is known about the interactions among these immune cells. Because of the delayed nature of secondary brain damage after ICH, adaptive immune cells may play an important role in the subacute and the regenerative phases after ICH.

No pharmacological interventions have demonstrated significant efficacy to improve functional outcomes after ICH; and less than one third of ICH patients attain functional independence by 6 months after ICH. As such, treatment options for ICH are limited, and there is an enormous unmet medical need for agents that may improve neurological recovery and reduce post-stroke disability.

Only a handful of clinical studies had investigated the effect of pharmacological agents, such as cyclo-oxygenase inhibitors (celecoxib), statins (rosuvastatin, simvastatin), PPAR-gamma agonist (pioglitazone) and iron chelators (deferoxamine) in the treatment of ICH. These studies, although providing valuable information on the particularities of the disease, did not result in any effective pharmacological therapy of ICH. Hence to date, no pharmaceutical treatment for ICH is available.

Two recent open label trials with another S1P-receptor modulator, fingolimod (Fu et al. JAMA Neurol. 2014; Fu et al. PNAS 2014), suggest an impact on edema formation and improved neurological outcome in ICH and ischemic stroke. In the study of fingolimod in ICH (JAMA Neurol. 2014; Fu et al. 2014) patients were assigned to receive standard management or standard management plus fingolimod at doses of 0.5 mg orally once daily for 3 consecutive days. The authors showed that while there were no differences in hematoma volume between the patients that received fingolimod and the patients that did not receive it, absolute PHE volume (aPHE) was lower in fingolimod-treated patients at Day 7 but not at Day 14; and relative PHE (rPHE; aPHE/hematoma volume) was significantly lower in fingolimod-treated patients at both Day 7 and Day 14 after ICH. However, limitations of the Fu et al. studies include lack of randomization, lack of placebo controls, and small sample sizes. Furthermore, fingolimod interacts with four of five known S1P receptors, i.e. S1P1, S1P3, S1P4 and S1P5.

As of today the inflammatory and immune mechanisms involved in stroke are not well understood. In addition targeting the highly dynamic events that occur during inflammation in the relatively inaccessible brain microenvironment is challenging, and an incomplete understanding of the interactions between the immune system and the brain during stroke limits progress.

There is therefore a high unmet need for an effective medicament for the treatment of stroke, in particular ICH, which at the same time has minimal or no side effects and good efficacy.

### SUMMARY OF THE INVENTION

The present disclosure provides a novel dosing regimen for the administration of siponimod, or pharmaceutically acceptable co-crystals, salts, hydrates, solvates, polymorphs and/or mixtures thereof, in the treatment of stroke, in particular hemorrhagic stroke, more in particular ICH.

Surprisingly it has been found that by administering siponimod according to the present novel dosing regimen for the treatment of stroke, it is possible to reduce the side effects which may be associated with the administration of siponimod, such as the negative chronotropic side effect affecting heart rate) and at the same time to produce a fast-acting anti-inflammatory effect to eliminate or reduce the inflammation processes and secondary injuries associated with stroke, in particular with ICH.

In particular the present disclosure provides a method of treatment of stroke, in particular of hemorrhagic stroke, more in particular of intracerebral hemorrhage (ICH) with siponimod, or pharmaceutically acceptable salts, hydrates, solvates, polymorphs, co-crystals and/or mixtures thereof, wherein siponimod is (a) intravenously administered to a human subject in need thereof multiple consecutive doses over a given time period, wherein
(i) the first administered dose is not less than 0.25 mg and not more than 1.25 mg; and wherein
(ii) each dose of the one or more consecutive doses administered after the first dose is not less than the directly preceding administered dose and not more than the directly subsequent administered dose; and wherein
(iii) the total sum of the consecutive doses administered over a time period of 24 consecutive hours is lower than the maintenance daily dose; and subsequently
(b) administering the maintenance daily dose of siponimod for a maintenance time period of at least 2 days, e.g. 3 days, wherein the maintenance daily dose is not less than 2 mg and not more than 20 mg of siponimod.

The maintenance daily dose of siponimod may be administered either parentally, e.g. via intravenous (i.v.) administration, or orally, e.g. tablets.

The present disclosure further provides a method of treatment of stroke in particular hemorrhagic stroke, more in particular ICH, with a combination comprising siponimod or pharmaceutically acceptable salts, hydrates, solvates, polymorphs, co-crystals and/or mixtures thereof, and one or more therapeutically active ingredients,

The present disclosure further provides the use of a new parenteral formulation of siponimod, which is liquid and preferably is administered intravenously (i.v. administration) in the treatment of stroke, in particular of hemorrhagic stroke, more in particular of intracerebral hemorrhage (ICH).

### BRIEF DESCRIPTION OF THE FIGURES

FIG. 1: Example of a dosing regimen schedule, wherein siponimod is administered 7 days i.v. with titration by 7 days p.o. (per os) and wherein the maintenance daily dose is 10 mg of siponimod.

FIG. 2: Summary of mean daily minimum heart rate of the dose titration study (from 0.25 mg to 10.0 mg) versus the daily fixed dose of 10.0 mg of siponimod over 12 Days.

FIG. 3: Simulated mean pharmacokinetic (PK) profiles of siponimod in *1/*1 subjects. First day of i.v. treatment (total daily dose of 1.75 mg) versus oral solid drug of 1.75 mg & 0.25 mg of siponimod. Flatter concentration-time curve slope for the i.v. dosing (2×0.25 mg/6 h) in the first 12 hours of infusion compared to that of an oral dose of 0.25 mg (starting dose of the oral dose-titration regimen). Similar concentration-time slope for the i.v. dosing (0.5 mg/6h and 0.75 mg/6h) in the second 12 hours of infusion. Higher slopes of the concentration-time curve after the first day are not expected to result in bradyarrhythmia as desensitization has mostly been completed.

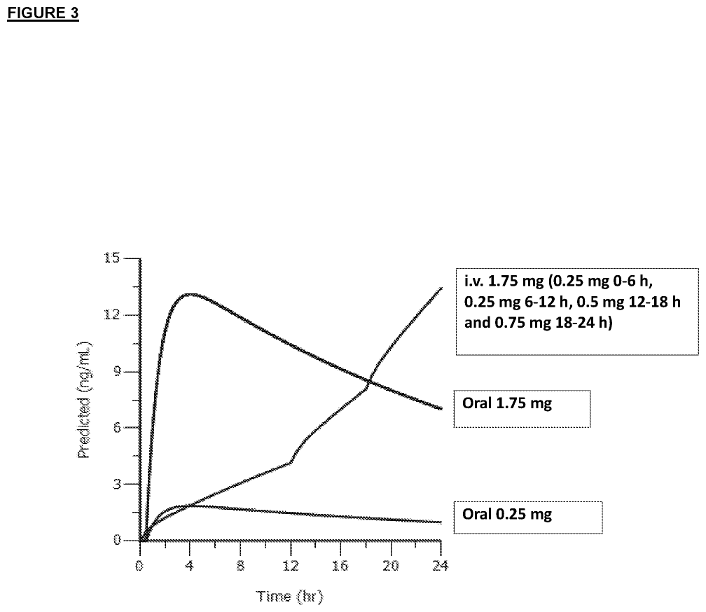

FIG. 4: Time course of perihematomal edema (PHE). Plateaus at 7-11 days after ICH (Staykov et al. Stroke 2011, 42:2625-2629). Time course of ICH and absolute PHE volumes (A) and relative PHE (B) in 90 patients with complete follow-up at all time points shown. Significant increase in absolute and relative PHE, as compared with the preceding value, is marked with asterisk. Error bars represent standard error (SE).

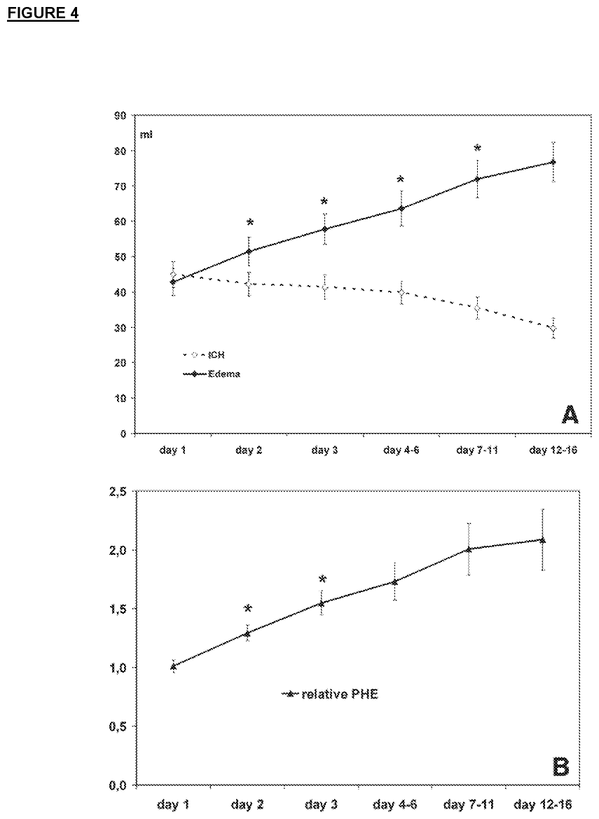

FIG. 5a: Simulated Absolute Lymphocyte Count (ALC) profiles of siponimod in *1/*1 subjects on the Day 1 of i.v. treatment versus oral drug substance containing 1.75 mg & 0.25 mg of siponimod. The new proposed Fibonacci i.v. titration reaching a Day-1 dose of 1.75 mg of siponimod, achieves a similar reduction in ALC on Day 1 than a 1.75 mg oral dose while efficiently mitigating the bradyarrhythmic effects during the initial treatment phase of step (a) of the present disclosure. Population of 1000 patients with weight normally distributed with mean 70.5 kg and standard deviation of 6, is simulated. Bioavailability is considered to be equal to 0.84 for this simulation. Shaded areas represent 95% Prediction Interval, bold line (i.v. treatment), dashed line (0.25 mg of siponimod) and dotted line (1.75 mg of siponimod) are means of the simulated population.

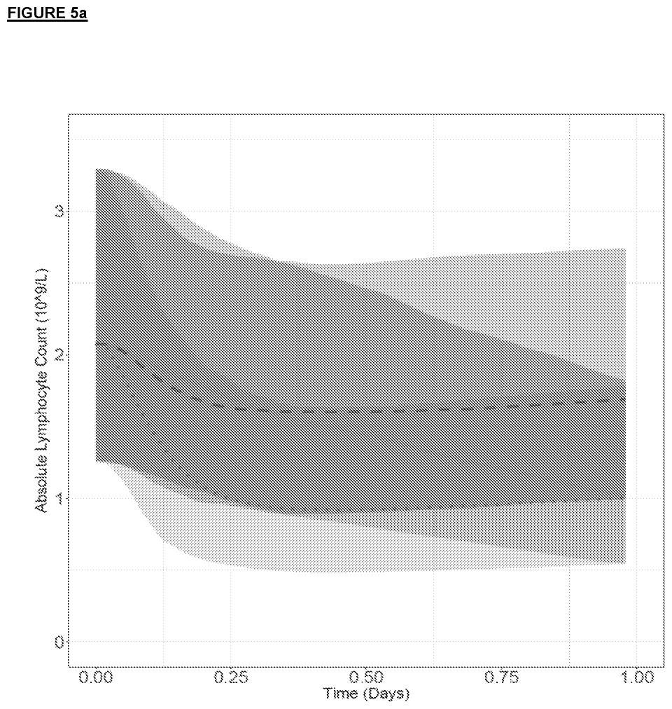

FIG. 5b: Simulated Absolute Lymphocyte Count (ALC) profiles of siponimod in *1/*1 subjects on Days 1-3 of i.v. titration up to reaching the target daily dose of siponimod of 10 mg (72 h) versus oral doses of 1.75 mg (Day 1), 8.25 mg (Day 2) and 10 mg (Day 3). Population of 1000 patients with weight normally distributed with mean 70.5 kg and standard deviation of 6, is simulated. Bioavailability is considered to be equal to 0.84 for this simulation. Shaded areas represent 95% Prediction Interval, bold line (i.v. treatment) and dashed line (oral treatment) are means of the simulated population.

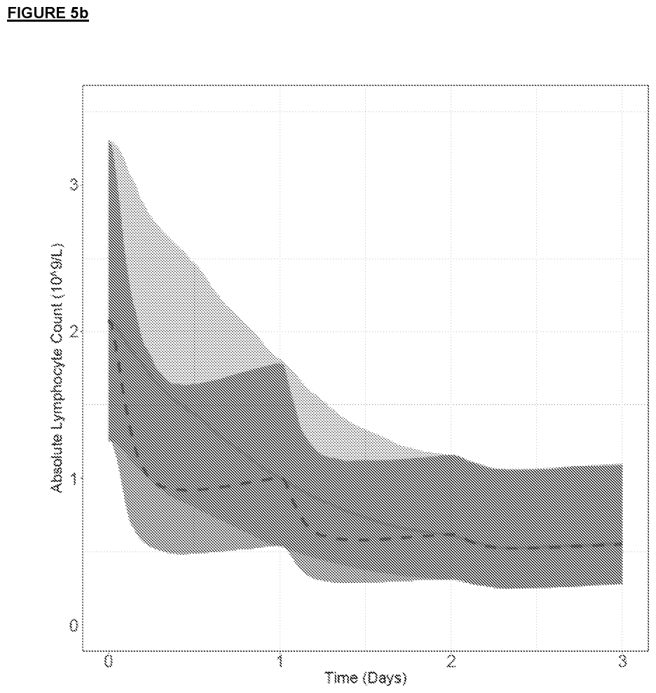

### DETAILED DESCRIPTION OF THE INVENTION

The dosing regimen of the present disclosure comprises a regimen for the initiation of siponimod therapy in a situation of clinical/medical emergency, such as a stroke event, in particular hemorrhagic stroke event, more in particular ICH event, which has the advantage of allowing a rapid achievement of the maintenance daily dose of siponimod, with minimal negative chronotropic effects, e.g. minimal or no transient bradycardia, sinus pauses (SPs) and/or AV blocks (AVB) effect associated with siponimod therapy.

Furthermore it has been surprisingly found that administering siponimod according to the novel dosing regimen of the present disclosure may significantly reduce, or even completely, eliminate the risk that the patient suffering from stroke, in particular hemorrhagic stroke, more particularly ICH, may (further) suffer from undesired heart effects associated with the use of siponimod, e.g. atrio-ventricular (AV) blocks or heart pauses or abrupt drop in heart rate, and at the same time prevents or minimizes the deleterious consequences of secondary injury or edema e.g., PHE formation and prevents or reduces physical, mental impairments such as paralysis or problems controlling movement, sensory disturbances including pain, problems using or understanding language, problems with thinking and memory, and/or emotional disturbances, resulting from ICH.

Surprisingly it has been found that administering siponimod according to the novel dosing regimen of the present disclosure also improves functional outcome in patient suffering from stroke, in particular suffering from hemorrhagic stroke, more in particular suffering from ICH, such as improving global functioning measured by the modified Rankin Scale (mRS) on Day 90 after ICH.

The novel dosing regimen of the present disclosure has the advantage of providing an early therapeutic treatment effect while timely desensitizing the system by S1 P receptor internalization and reducing GIRK activation (i.e., activation of the G protein-coupled inwardly-rectifying potassium channels) without provoking the bradyarrhythmia (e.g., subthreshold desensitization) which may be associated with the administration of siponimod.

Furthermore the novel dosing regimen of the present disclosure also permits to administer siponimod to categories of patients for which the risk/benefit ratio may otherwise be less favorable. Such patients could for example include patients which are CYP2C9*2*3 and CYP2C9*3*3 poor metabolizers.

### Rationale for Dosing Regimen, Route of Administration and Duration of Treatment

The safety profile of siponimod includes the following identified risks: (i) bradyarrhythmia (including first dose negative chronotropic effects and AV blocks), (ii) liver enzyme elevation, such as transaminase elevation and (iii) lymphopenia due to lymphocyte redistribution (main targeted pharmacodynamic (PD) effect of siponimod). However the (ii) liver transaminase elevation and (iii) lymphopenia risks are considered monitorable/manageable even under higher exposure levels for the relative short-term treatment of patient suffering from stroke, in particular suffering from hemorrhagic stroke, more in particular suffering from ICH. Therefore (i) bradyarrhythmia remains the most relevant of the adverse event (AE) to keep under control during the treatment of stroke.

Siponimod is a potent and selective S1P1/S1P5 receptor modulator and has an initial transient negative chronotropic and dromotropic (conduction speed in the AV node, and subsequently the rate of electrical impulses in the heart) effects both in healthy subjects and MS patients. These negative chronotropic and dromotropic effects are expected to affect stroke patients as well. Pronounced bradycardia may be associated with bradyarrhythmia (e.g. AV blocks, AVB, and sinus pauses, SP). While such bradycardia and its potential related side-effects might not be highly problematic for healthy patients, it might be critical for subjects suffering from stroke, which are a particularly fragile and life threatened patient population. Thus a thorough valuation on how to use safely siponimod in patient suffering from stroke is required. At the same time, an efficacious treatment of stroke, i.e. an efficacious prevention and/or management of the immune and inflammatory components associated to a stroke event, demands siponimod to act quickly and provide a rapid therapeutic effect. Therefore a dosing regimen which balances both efficacy and safety is to be developed.

Hence, the rationale underlying the novel dosing regimen of siponimod in stroke is based on a balance of neurological, clinical efficacy and safety considerations, which are summarized hereinafter.

Based on the results of a siponimod single dose study (SAD study) in healthy volunteers, the single maximum tolerated dose (single MTD) in healthy subjects was determined to be 25 mg. The 25 mg single dose showed a favorable safety and tolerability profile.

In another study in healthy subjects siponimod was administered in multiple doses, i.e. 0.3, 1, 2.5, 10 and 20 mg, over 28 days (multiple ascending dosing study, MAD study). The maximum investigated multiple dose of 20 mg of siponimod was determined to be associated with symptomatic bradycardia as the only relevant adverse event.

S1P receptor modulators, as mentioned above, are known to cause dose dependent transient decrease in heart rate within 2-3 hours of drug intake (Legangneux et al. 2012, Hoch et al. 2014). In order to evaluate on how to best mitigate the bradyarrhythmic risk of siponimod, a multiple-dose titration clinical study in healthy subjects was run.

The primary objective of this study was to measure the daily chronotropic effects of two siponimod dose-titration regimens (from 0.25 mg to 10 mg; Dose Titration (DT) 1# and 2#, respectively) compared to daily chronotropic effects of oral siponimod 10 mg (fixed dose, once daily) and placebo over 12 days. The heart rate (HR) changes have been compared between subjects exposed to 10 mg siponimod once daily (QD) with or without 2 different up-titration schemes. The titration scheme of this study was:
DT 1#: Day 1: 0.25 mg; Day 2: 0.25 mg; Day 3: 0.25 mg; Day 4: 0.5 mg; Day 5: 1.0 mg; Day 6: 2.0 mg; Day 7: 4.0 mg; Day 8: 8.0 mg and from Day 9 to Day 12: 10.0 mg daily.
DT 2#: Day 1: 0.25 mg; Day 2: 0.25 mg; Day 3: 0.5 mg; Day 4: 0.75 mg; Day 5: 1.25 mg; Day 6: 2.0 mg; Day 7: 3.0 mg; Day 8: 5.0 mg; from Day 9 to Day 12: 10.0 mg daily.

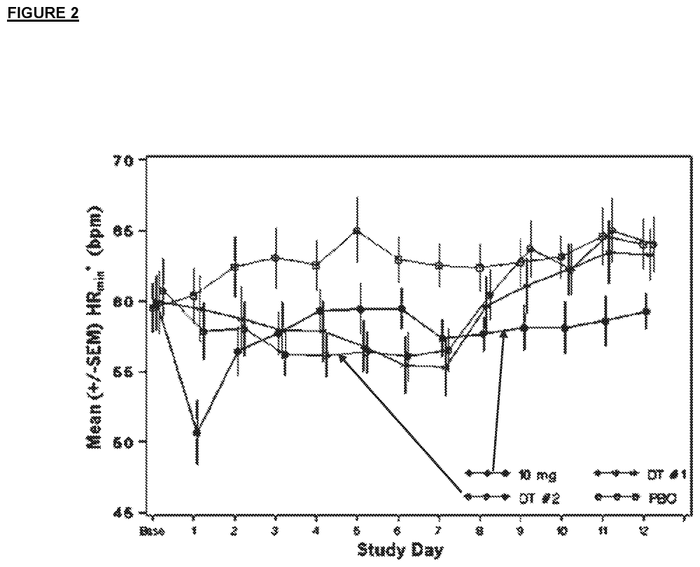

Neither DT 1# nor DT 2# resulted in clinically significant bradycardia or AV conduction effects. Both titration regimens showed a favorable treatment difference on each of Days 1-12 versus the non-titration regimen on Day 1 for heart rate effects. Heart rates in the non-titration regimen showed considerable separation from placebo throughout the study (FIG. 2). There was no statistically significant reduction in heart rate vs. placebo on Day 1 in either titration regimen. On Days 3 to 7 subjects in DT 1# and DT 2# experienced minor reductions in HR. By Day 9, heart rates in both titration regimens were comparable to placebo. This effect was maintained until end of treatment on Day 12. The starting dose of 0.25 mg of the 2 tested DT regimens was not associated with bradyarrhythmia. It was then concluded that both titration regimens effectively attenuated the initial bradycardia observed on Day 1 of treatment with siponimod 10 mg daily fixed-dose.

In a phase II dose finding study of siponimod in patients with relapsing-remitting multiple sclerosis (CBAF312A2201) safety, tolerability and efficacy on magnetic resonance imaging (MRI) brain lesion parameters were evaluated. The dose response curve for the MRI-based efficacy of siponimod given orally once daily compared with placebo was determined.

Siponimod dose level 10 mg appeared to contribute little additional efficacy compared to 2 mg, and appeared to have a worse safety profile. Siponimod 1.25 mg to 2 mg dose range appeared to be close to maximal efficacy, with a good safety profile. The dose-response curve as defined by the primary endpoint, favored the upper part of this range, i.e. a dose in the range of siponimod 2 mg, since efficacy seemed to decrease with lower doses. Siponimod achieved positive results in a clinical trial for the treatment of RRMS patients (Selmaj et al., Lancet Neurol, 2013, 12, 756-767) and is currently being investigated in an ongoing phase III study (EXPAND) in patients with SPMS. The dose of 2 mg of siponimod was the chosen dose for this follow up phase III study.

Siponimod achieved positive results in a clinical trial for the treatment of RRMS patients (Selmaj et al., Lancet Neurol, 2013, 12, 756-767) and is currently being investigated in an ongoing phase III study (EXPAND) in patients with SPMS. The dose of 2 mg of siponimod was chosen for this follow up phase III study and a five day uptitration was implemented.

Differently from MS, which is a chronic disease, stroke is an acute, life-threatening event that requires an immediate therapeutically effective intervention to prevent or at least minimize the post-stroke inflammatory/immunological cascade which may cause serious post-stroke physical and cognitive disorders. The titrations schemes of the clinical trial mentioned above, although safe, may not allow reaching the high dose required to impact the pathophysiology of stroke, e.g. ICH, quickly enough. The treatment of a patient suffering from stroke needs to be not only safe but also effective in a short lapse of time from the onset of the stroke. A titration time period of 8 days as in the healthy volunteer multiple-dose titration clinical study mentioned above, or even a titration period of 5 days as in the dosing regimen used in the phase III clinical trial in MS, would not be fast enough to ensure an efficacious treatment for patients suffering from stroke, in particular suffering from ICH.

A method of treatment which may minimize the negative effects of the secondary injury following stroke, in particular ICH, shall be a treatment which can quickly provide the subject suffering from stroke with a high exposure to siponimod by administered it in high dose within the shortest time period from the onset of ICH.

Amongst the various potential factors which may contribute to the reduction of inflammation, and which may therefore contribute to the efficacy of a treatment by siponimod, one important factor is the reduction of the Absolute Lymphocyte Count (ALC), being ALC known to play an important role in the inflammatory processes, including those in the brain. The exact mechanism by which an S1P receptor modulation may mitigate stroke pathophysiology is still not fully elucidated and thus, besides the Absolute Lymphocyte Count related effect (ALC-related effect), other potential mechanisms may play a role.

In the above mentioned multiple ascending dosing study in healthy volunteers, it has been shown that acute responses between the 0.3 mg dose and the 10 mg dose of siponimod on Day 1 of treatment showed a dose-dependent decline of ALC. Chronic responses showed that ALC decrease was dose and time-dependent, plateauing at about 80% at 10 mg, while 2.5 mg shows a lower reduction, close to 70% (Table 1).

TABLE 1

| BAF312 | % Emax0-12 h from | % Emax0-12 h from |
|---|---|---|
| Dose Group | Day 1 pre-dose | time-matched Day −1 |
| 0 mg (Placebo) | 8.7% | 22.5% |

| | | | |
|---|---|---|---|
| 0.3 | mg | 41.9% | 47.8% |
| 1 | mg | 71.3% | 73.8% |
| 2.5 | mg | 66.9% | 67.5% |
| 10 | mg | 83.7% | 86.1% |
| 20 | mg | 84.8% | 86.5% |

Based on the above series of considerations, a maintenance daily dose of 10 mg is especially suitable to demonstrate the effect of siponimod in the treatment of stroke, in particular in the treatment of ICH.

Clinical data have shown that bradyarrhythmic effects of siponimod are better correlated to the rate at which Cmax is achieved (i.e., concentration-time slope) than to AUC or Cmax. During the first 12-24 hours of the treatment it was hence considered beneficial to improve the safety of the treatment by reducing Cmax while delaying Tmax and mimic an oral dose of 0.25 mg of siponimod which represents the starting dose of the established oral dose titration regimen and demonstrated to be free of bradyarrhythmic effects.

In addition, clinical data suggest that most of the desensitization via internalization of the cardiac S1P receptor occurred during the first 12-24 hours. This was in line with clinical observations showing that bradyarrhythmic events mainly occur in the first 24 hours of treatment.

The above studies showed that it is possible to minimize the bradycardia by slowly increasing the dose and at the same time use an oral dose of 10 mg.

A comparison of bradyarrhythmic effects (HR, AVB, SP) of a 1 mg oral dose from previous studies and a 1 mg/day i.v. dose infused over 24 h in healthy subjects supported the hypothesis that such effects are related to the slope of the concentration-time curve.

In said study with siponimod administered intravenously, PK and PD were determined. The i.v. infusions of a single dose of 0.25 mg over 3 hours and of 1 mg over 24 hours (4×0.25 mg/6 hours infusion) of siponimod displayed an excellent cardiac safety profile. This finding was consistent with previous oral administrations of 0.25 mg (starting dose of the oral initial period dosing regimen). The results of this clinical study were the following:
a) The magnitude of PD (ALC) effects %Eₘₐₓ of i.v. of 0.25 mg dose over 3 hour and 1 mg dose over 24 hours was comparable to the effects after oral doses at the same dose levels in this study (0.25 mg) and previous clinical studies (1 mg).
b) The orally administered siponimod displayed good bioavailability (the oral bioavailability F% was 84%).
c) Median oral siponimod Tₘₐₓ was observed 8 hours after dosing.
d) Median i.v. siponimod Tₘₐₓ was observed at the end of the 3 hour and 24 hour infusions.
e) Geometric mean oral siponimod Cmax was -48% lower than mean i.v. siponimod
f) The route of administration did not alter the terminal T112 (between approximately 27 hours and 33 hours).
g) Siponimod exhibits dose linear and time-independent pharmacokinetics (PK).

Starting from the findings of the above mentioned clinical trials the new and inventive dosing regimen of the present disclosure was designed. Based on the above findings, the inventors of the novel present dosing regimen set the lower threshold of the first administered dose, i.e. first dose, to be not less than 0.25 mg of siponimod and the maintenance daily dose to be not less than 2 mg of siponimod.

In addition to the previously described benefits, the dosing regimen of the present disclosure has also the advantage to highly reduce the additional risks run by the CYP2C9 poor metabolizer. It is known that in humans, siponimod is eliminated from the systemic circulation due to metabolism (mainly by CYP2C9, followed by CYP3A4). With regard to the CYP2C9 metabolism of siponimod, another clinical study investigating siponimod pharmacokinetics (PK) parameters in poor metabolizers, it was experimentally determined that AUC of siponimod was approximately 2-fold and 4-fold when compared with reference AUC of extensive metabolizers (=CYP2C9*1*1 genotype), while _{Cmax} was only slightly greater in poor metabolizers (=CYP2C9*2*3 and CYP2C9*2*3 genotypes) and Tₘₐₓ was comparable in poor and extensive metabolizers.

As already described above, a stroke event is a clinical/medical emergency. In order to prevent or minimize the deleterious consequences of the secondary injury consequent to stroke, e.g. ICH, in a large patient population, a quick and strong intervention, i.e. the administration of a high dose of siponimod, possibly close to the maximum tolerated dose (MTD), may be crucial.

The dosing regimen of the present disclosure comprises a modified Fibonacci i.v. dose titration phase which has the advantage of allowing a rapid achievement of a 10 mg maintenance daily dose of siponimod, with minimal negative chronotropic effects.

The maintenance daily dose such as a 10 mg maintenance daily dose is a high dose which is efficacious and at the same time is well tolerated by weakened subject as the patients suffering from stroke, e.g. ICH, as well as by patient suffering from stroke, e.g. ICH, which are furthermore poor metabolizer. Indeed, because of the acute nature of this disease and the need for rapid intervention to interrupt early pathophysiological events taking place in ICH, it is not possible to stratify patients on entry or dose adjust patients within the total treatment window based on CYP2C9 genotyping, which typically takes >14 days to obtain. As such, it is not possible to exclude CYP2C9*3*3 patients, which make up <1% of the general population, from this acute treatment study. The risks of siponimod exposures in a patient subpopulation above MAD maximum levels for a short duration are outweighed by the severity of ICH and its sequelae.

The i.v. dosing regimen schedule of the present disclosure and intensive care unit monitoring mitigate the most serious adverse events (AEs), i.e., bradyarrhythmias; and the remaining prevalent AEs of headache, dizziness, and nasopharyngitis are not significant in an ICH population in an acute Stroke Unit/ICU setting, and resolve fully after discontinuation of drug.

More specifically the use of siponimod in the treatment of ICH according to the novel dosing regimen of the present disclosure allows preventing or minimizing the neurological and other clinical damages due to a cascade of inflammatory processes produced after the intracerebral hemorrhage (ICH) and it is safe. The administration of siponimod according to the dose regime of the disclosure further allows the patient to be quickly exposed at a high dose of siponimod and for (at least) the duration of the rise in perihematomal edema (PHE), which is considered to contribute to mass effect in the brain, to cause acute neurologic deterioration in patients, and even to be associated with poor long-term functional outcomes.

### Dosing Regimen

The present disclosure provides a novel dosing regimen which is adapted to prevent or minimize the deleterious consequences after stroke, in particular the secondary injury or edema formation, e.g. perihematomal edema formation, after ICH, and to eliminate or reduce the side effects which may be associated with the administration of siponimod, such as the negative chronotropic side effect or other heart effects.

### Secondary Injury

Secondary Injury is a term applied to the destructive and self-propagating biological changes in cells and tissues that lead to their dysfunction or death over hours or weeks after the initial insult (the “primary injury”). In stroke the initial injury is usually mechanical. For instance, in ICH the primary brain injury is produced through direct mechanical effects of the hemorrhage. The produced hematoma cleaves or dissects neuronal tissue over several hours leading to the presenting symptoms. The loss of neurons within the hematoma is extensive; although some islands of preserved neurons may exist. Hematoma expansion (HE) stops due to tamponade effect of the surrounding tissue or clotting at the parent vessel and leads to secondary brain injury through processes that evolve over hours to days. Due to the rapid onset, the primary injury of ICH is difficult to treat. The best way to minimize the injury from primary insult would be the preventative measures in high-risk individuals and early access to care once symptoms start. Primary injury, as it is mentioned above, is followed by secondary injury in the perihemorrhagic region over several days to weeks and provides a longer treatment window than the primary injury.

In ICH the severe disability and/or death and related poor outcome is due to the combined effect of primary injury and secondary injury. There are multiple factors that contribute to secondary injury after ICH, some of which may occur concurrently or in sequence. Secondary injury after ICH can be separated into two major types: rebleeding causing hematoma expansion (HE) and the consequences of repair pathways along the continuum of neuroinflammation and neuronal death, including perihematomal edema (PHE), perihematomal tissue damage, including breakdown of the blood-brain barrier (BBB), elevated intracranial pressure (ICP), hydrocephalus, and brain atrophy.

It is believed that the destructive phase of secondary injury is the main responsible for cell death functional deficits and consequent neurological deterioration after ICH.

### Edema and Perihematomal Edema (PHE)

In ICH there are two predominant mechanisms of brain injury from intracerebral hemorrhage: mechanical injury from the primary hematoma (including growth of that hematoma, e.g. hematoma expansion (HE)), and secondary injury from perihematomal edema and inflammation.

The acute mortality and morbidity in spontaneous ICH have been linked to early hematoma expansion, cytotoxic and vasogenic perihematomal edema (PHE) formation, reduction in cerebral perfusion pressure, and raised intracranial pressure (ICP). It is widely accepted that hematoma-induced neuronal damage is irreversible while that from PHE is reversible which makes the latter a potential therapeutic target. Hematoma volume is known to be a powerful predictor of outcome and also an independent predictor of absolute PHE volume (Jauch E, Kothari R, et al. Stroke. 1999).

The pathogenesis of PHE is related primarily to three processes: hematoma expansion and increased blood barrier permeability, clot retraction and activation of coagulation pathway and, finally, cytotoxic edema caused by erythrocyte lysis and hemoglobin breakdown products. In ICH the PHE plateaus between days 7 and 16 after ICH. More precisely, the “absolute PHE” (aPHE) plateaus between days 11 and 16 after ICH and the “relative PHE” (rPHE), which provides a more predictive measure for the ICH outcome, plateaus between days 11 and 14 after ICH (Staykov et al. 2011). PHE is widely agreed to contribute to long-term sequelae after ICH. PHE can contribute to mass effect, cause acute neurologic deterioration in patients, and has even been associated with poor long-term functional outcomes.

Heart effects

Heart effects are for instance heart rate reduction, transient bradycardia, chronotropic or dromotropic effects, including AV blocks, which include first degree AV blocks (e.g. PR intervals greater than 0.2 seconds) and second degree AV blocks e.g. first degree AV blocks. Heart effects include sinus pauses, e.g. sinus pauses greater than 2 seconds.

### Embodiments of the present disclosure

According to the disclosure, the following embodiments are provided:
Embodiment 1.1: A method of treating stroke in a human subject suffering from stroke said method comprising
(a) intravenously administering to said subject multiple consecutive doses of siponimod over a time period equal to or up to 96 hours calculated starting at the first intravenously administered dose, wherein
(i) the first administered dose is not less than 0.25 mg and not more than 1.25 mg; and wherein
(ii) each dose of the one or more consecutive doses administered after the first dose is not less than the directly preceding administered dose and not more than the directly subsequent administered dose; and wherein
(iii) the total sum of the consecutive doses administered over a time period of 24 consecutive hours is lower than the maintenance daily dose; and subsequently
(b) administering a maintenance daily dose of siponimod for a maintenance time period of at least 2 days, wherein
(i) the maintenance daily dose is not less than 2 mg and not more than 20 mg of siponimod.
Embodiment 1.2: A method of treating stroke in a human subject as defined in the embodiment 1.1, wherein the intravenous administration to said subject of the multiple consecutive doses of siponimod according to step (a) is done over a time period equal to or up to 72 hours calculated starting at the first intravenously administered dose.
Embodiment 1.3: A method of treating stroke in a human subject as defined in the embodiment 1.1 or 1.2, wherein the intravenous administration to said subject of the multiple consecutive doses of siponimod according to step (a) is done over a time period equal to or up to 48 hours calculated starting at the first intravenously administered dose.
Embodiment 1.4: A method of treating stroke in a human subject as defined in any of the embodiment 1.1 to 1.3, wherein the intravenous administration to said subject of the multiple consecutive doses of siponimod according to step (a) is done over a time period equal to or up to 24 hours calculated starting at the first intravenously administered dose.
Embodiment 1.5: A method of treating stroke in a human subject as defined in any of the embodiments 1.1 to 1.4, wherein the first administered dose of step (a) is 0.25 mg.
Embodiment 1.6: A method of treating stroke in a human subject as defined in any of the embodiments 1.1 to 1.4, wherein the first administered dose of step (a) is 0.5 mg.
Embodiment 1.7: A method of treating stroke in a human subject as defined in any of the embodiments 1.1 to 1.4, wherein the first administered dose of step (a) is 0.75 mg.
Embodiment 1.8: A method of treating stroke in a human subject as defined in any of the embodiments 1.1 to 1.4, wherein the first administered dose of step (a) is 1.0 mg.
Embodiment 1.9: A method of treating stroke in a human subject as defined in any of the embodiments 1.1 to 1.4, wherein the first administered dose of step (a) is 1.25 mg.
Embodiment: 1.10: A method of treating stroke in a human subject as defined in any of the embodiments 1.1 to 1.9, wherein the maintenance daily dose of step (b)(i) is not less than 2 mg and not more than 15 mg of siponimod.
Embodiment 1.11: A method of treating stroke in a human subject as defined in any of the embodiments 1.1 to 1.10, wherein the maintenance daily dose of step (b)(i) is not less than 2 mg and not more than 10 mg of siponimod.
Embodiment 1.12: A method of treating stroke in a human subject as defined in any of the embodiments 1.1 to 1.11, wherein the maintenance daily dose of step (b)(i) is not less than 2 mg and not more than 5 mg of siponimod.
Embodiment 1.13: A method of treating stroke in a human subject as defined in any of the embodiments 1.1 to 1.9, wherein the maintenance daily dose of step (b)(i) is 20 mg of siponimod.
Embodiment 1.14: A method of treating stroke in a human subject as defined in any of the embodiments 1.1 to 1.10, wherein the maintenance daily dose of step (b)(i) is 15 mg of siponimod.
Embodiment 1.15: A method of treating stroke in a human subject as defined in any of the embodiments 1.1 to 1.11, wherein the maintenance daily dose of step (b)(i) is 10 mg of siponimod.
Embodiment 1.16: A method of treating stroke in a human subject as defined in any of the embodiments 1.1 to 1.12, wherein the maintenance daily dose of step (b)(i) is 5 mg of siponimod.
Embodiment 1.17: A method of treating stroke in a human subject as defined in any of the embodiments 1.1 to 1.12, wherein the maintenance daily dose of step (b)(i) is 2 mg of siponimod.
Embodiment 1.18: A method of treating stroke in a human subject as defined in any of the embodiments 1.1 to 1.17, wherein the maintenance daily dose of siponimod administered in step (b) is administered for a maintenance time period of at least 3 days, e.g. for a maintenance time period of 3 or 4 days.
Embodiment 1.19: A method of treating stroke in a human subject as defined in any of the embodiments 1.1 to 1.18, wherein the maintenance daily dose of siponimod administered in step (b) is administered for a maintenance time period of at least 5 days, e.g. for a maintenance time period of 5 days.
Embodiment 1.20: A method of treating stroke in a human subject as defined in any of the embodiments 1.1 to 1.19, wherein the maintenance daily dose of siponimod administered in step (b) is administered for a maintenance time period of at least 7 days, e.g. for a maintenance time period of 12 days.
Embodiment 1.21: A method of treating stroke in a human subject as defined in any of the embodiments 1.1 to 1.20, wherein the maintenance daily dose of siponimod administered in step (b) is administered for a maintenance time period of at least 14 days, e.g. for a maintenance time period of 14 days.
Embodiment 1.22: A method of treating stroke in a human subject as defined in any of the embodiments 1.1 to 1.21, wherein the daily maintenance dose of siponimod administered in step (b) is administered for a maintenance time period of at least 21 days.
Embodiment 1.23: A method of treating stroke in a human subject as defined in any of the embodiments 1.1 to 1.22, wherein the maintenance daily dose of siponimod administered in step (b) is administered for a maintenance time period of at least 28 days.
Embodiment 1.24: A method of treating stroke in a human subject as defined in any of the embodiments 1.1 to 1.23, wherein the maintenance daily dose of siponimod administered in step (b) is administered for a maintenance time period of at least 35 days.
Embodiment 1.25: A method of treating stroke in a human subject as defined in any of the embodiments 1.1 to 1.24, wherein the administration of the maintenance daily dose of siponimod in step (b) comprises intravenous administration.
Embodiment 1.26: A method of treating stroke in a human subject as defined in any of the embodiments 1.1 to 1.25, wherein the administration of the maintenance daily dose of siponimod in step (b) comprises oral administration.
Embodiment 1.27: A method of treating stroke in a human subject as defined in any of the embodiments 1.1 to 1.26, wherein the administration of the maintenance daily dose of siponimod in step (b) is carried out in a first phase by intravenous administration and in a second phase by oral administration, preferably the first phase has a duration of 5 days and the second phase has a duration of 7 days.
Embodiment 1.28: A method of treating stroke in a human subject as defined in any of the embodiments 1.1 to 1.27, said method further comprising
(c) continuously monitoring said subject via cardiovascular telemetry for at least the first 24 hours, preferably for at least the first 48 hours calculated starting from the administration of the first dose of siponimod.
Embodiment 1.29: A method of treating stroke in a human subject as defined in any of the preceding embodiments 1.1. to 1.28, wherein if a consecutive dose in step (a) is increased by an increment, said increment is governed by a modified Fibonacci series, i.e. a given dose is the sum of two directly previous doses ±40%, for example ±35%, for example ±30%, for example ±20%, e.g. about ±23%, or for example ±10%.
Embodiment 1.30: A method of treating stroke in a human subject, as defined in any of the embodiments 1.1, 1.3, 1.5, 1.10, 1.11, 1.15 or from 1.18 to 1.29, comprising
(a) intravenously administering to said subject multiple consecutive doses of siponimod over a time period equal to or up to 48 hours calculated starting at the first intravenously administered dose, wherein
on Day 1, the administered doses are 0.25 mg over 6 hours, then 0.25 mg over 6 hours, then 0.5 mg over 6 hours, and then 0.75 mg over 6 hours for a total Day 1 dose of 1.75 mg; and
on Day 2, the administered doses are 1.25 mg over 6 hours, then 2 mg over 6 hours, then 2.5 mg over 6, and then 2.5 mg over 6 hours for a total Day 2 dose of 8.25 mg; and
(b) intravenously administering a maintenance daily dose of 10 mg of siponimod on Day 3 through Day 7; and optionally orally administering the maintenance daily dose of 10 mg of siponimod on and after Day 8, preferably on Day 8 through Day 14; and
wherein said method further optionally comprises
(c) continuously monitoring said subject via cardiovascular telemetry for at least the first 24 hours, preferably for at least the first 48 hours, calculated starting from the administration of the first dose of siponimod.
Embodiment 1.31: A method of treating stroke in a human subject as defined in the embodiment 1.1 to 1.30, wherein when in step (b) siponimod is administered orally it is administered in the form of an oral solid dosage form.
Embodiment 1.32: A method of treating stroke in a human subject as defined in the Embodiment 1.31, wherein the oral solid dosage form of step (b) is an immediate release oral solid dosage form.
Embodiment 1.33: A method of treating stroke in a human subject as defined in the embodiment 1.32, wherein the oral immediate release solid dosage form administered in step (b) is in the form of tablets having the composition as provided in Table 2.1 or Table 2.2.
Embodiment 1.34: A method of treating stroke in a human subject as defined in any of the embodiments 1.31 to 1.33, wherein the 10 mg maintenance daily dose of siponimod of step (b) is administered to the human subject in need thereof in the form of
   (a) 5 tablets of 2 mg strength; or
   (b) 2 tablets of 5 mg strength; or
   (c) 1 tablet of 10 mg strength;
and wherein when the dose is administered by more than 1 tablet, the tablets are administered simultaneously, sequentially or separately, preferably simultaneously.
Embodiment 1.35: A method of treating stroke in a human subject, preferably hemorrhagic stroke, more preferably ICH, as defined in any of the preceding embodiments 1.1 to 1.34, wherein the administered i.v. composition containing siponimod is obtained by diluting, for example in saline or 5% glucose solution, a concentrate containing siponimod, wherein said concentrate
   (i) is in the form of a liquid;
   (ii) contains 1 mg/mL of siponimod; and
   (iii) contains
      7 wt. %-13 wt. % of 2-hydroxypropyl-f3-cyclodextrin (HPBCD);
      a buffer agent; and
      optionally a tonicity agent.
Embodiment 1.36: A method of treating stroke in a human subject as defined in the embodiment 1.35, wherein stroke is preferably hemorrhagic stroke, more preferably ICH and wherein the administered i.v. composition containing siponimod is obtained by diluting, for example in saline or 5% glucose solution, a concentrate containing siponimod, wherein said concentrate
   (i) is in the form of a liquid; and
   (ii) contains 1 mg/mL of siponimod; and in addition
   (iii) contains
      10 wt. % of 2-hydroxypropyl-f3-cyclodextrin (HPBCD);
      3 wt. % mannitol; and
      0.06 wt. % 2-amino-2-(hydroxymethyl)propan-1,3-diol (Tris); and
   (iv) its pH is about 8.
Embodiments 1.37: A method of treating stroke in a human subject as defined in any of the preceding embodiments 1.1 to 1.36, wherein stroke is preferably ICH and wherein the first dose of said method is administered within 72 hours, preferably within 48 hours, more preferably within 24 hours, e.g. within 6 or 12 hours, from the onset of ICH.
Embodiment 1.38: A method of treating stroke in a human subject as defined in any of the preceding embodiments 1.1 to 1.37, wherein stroke is intracerebral hemorrhagic stroke (ICH).
Embodiment 1.39: A method of treating stroke in a human subject as defined in any of the preceding embodiments 1.1 to 1.38, wherein stroke, e.g. ICH, is grade 4 stroke or higher as defined by the National Institute of Health Stroke Scale (NIHSS).
Embodiment 1.40: A method of treating stroke in a human subject as defined in any of the preceding embodiments 1.1 to 1.39, wherein stroke, e.g. ICH, is grade 6 stroke or lower as defined by the National Institute of Health Stroke Scale (NIHSS).
Embodiments 1.41: A method of treating stroke in a human subject as defined in any of the preceding embodiments 1.1 to 1.40, wherein stroke is a spontaneous intracerebral haemorrhage (SICH), wherein the supratentorial intracerebral hemorrhage is in deep brain structures (putamen, thalamus, caudate, and associated deep white matter tracts) with a volume 10 mL but 30 mL calculated by the ABC/2 method) determined by routine clinical magnetic resonance imaging (MRI) or computerized tomography (CT).
Embodiments 1.42: A method of treating stroke, e.g. ICH, in a human subject, as defined in any of the preceding embodiments 1.1 to 1.41, wherein said subject has a Glasgow Coma Scale (GCS) motor score which is no less than 6.
Embodiment 1.43: A method of treating stroke, e.g. ICH, in a human subject, as defined in any of the preceding embodiments 1.1 to 1.42, wherein the subject is a CYP2C9*2*3 poor metabolizer or a CYP2C9*3*3 poor metabolizer.
Embodiment 1.44: A method of treating stroke, e.g. ICH, in a human subject, as defined in any of the preceding embodiments 1.1 to 1.43, wherein siponimod is contained in an oral solid dosage form and is in the form of a co-crystal with fumaric acid.
Embodiment 1.45: A method of improving global functioning of human subject suffering from stroke, in particular suffering from ICH, measured by the modified Rankin Scale (mRS) on Day 90 after ICH to achieve a mRS score equal to 0, 1 or 2, wherein the administration of siponimod is according to the method of treating a human subject having stroke as defined in any of the preceding embodiments 1.1 to 1.44.
Embodiment 2.1: Siponimod for use in the treatment of stroke in a human subject suffering from stroke, wherein
(a) multiple consecutive doses of siponimod are intravenously administered to said subject over a time period equal to or up to 96 hours calculated starting at the first intravenously administered dose, wherein
(i) the first administered dose of siponimod is not less than 0.25 mg and not more than 1.25 mg; and wherein
(ii) each dose of the one or more consecutive doses of siponimod which are administered after the first dose is not less than the directly preceding administered dose and not more than the directly subsequent administered dose; and
(iii) the total sum of the consecutive doses of siponimod administered over a time period of 24 consecutive hours is lower than the maintenance daily dose; and wherein subsequently
(b) the maintenance daily dose of siponimod is administered for a maintenance time period of at least 2 days, wherein
(i) said maintenance daily dose is not less than 2 mg and not more than 20 mg of siponimod.
Embodiment 2.2: Siponimod for use in the treatment of stroke in a human subject according to the embodiment 2.1, wherein the multiple consecutive doses of siponimod are intravenously administrated to said subject according to step (a) over a time period equal to or up to 72 hours calculated starting at the first intravenously administered dose.
Embodiment 2.3: Siponimod for use in the treatment of stroke in a human subject according to any of the embodiments 2.1 or 2.2, wherein the multiple consecutive doses of siponimod are intravenously administrated to said subject according to step (a) over a time period equal to or up to 48 hours calculated starting at the first intravenously administered dose.
Embodiment 2.4: Siponimod for use in the treatment of stroke in a human subject according to any of the embodiments 2.1 to 2.3, wherein the multiple consecutive doses of siponimod are intravenously administrated to said subject according to step (a) over a time period equal to or up to 24 hours calculated starting at the first intravenously administered dose.
Embodiment 2.5: Siponimod for use in the treatment of stroke in a human subject according to any of the embodiments 2.1 to 2.4, wherein the first administered dose of step (a) is 0.25 mg of siponimod.
Embodiment 2.6: Siponimod for use in the treatment of stroke in a human subject according to any of the embodiments 2.1 to 2.4, wherein the first administered dose of step (a) is 0.5 mg of siponimod.
Embodiment 2.7: Siponimod for use in the treatment of stroke in a human subject according to any of the embodiments 2.1 to 2.4, wherein the first administered dose of step (a) is 0.75 mg of siponimod.
Embodiment 2.8: Siponimod for use in the treatment of stroke in a human subject according to any of the embodiments 2.1 to 2.4, wherein the first administered dose of step (a) is 1.0 mg of siponimod.
Embodiment 2.9: Siponimod for use in the treatment of stroke in a human subject according to any of the embodiments 2.1 to 2.4, wherein the first administered dose of step (a) is 1.25 mg of siponimod.
Embodiment 2.10: Siponimod for use in the treatment of stroke in a human subject to any of the embodiments 2.1 to 2.9, wherein the maintenance daily dose of step (b)(i) is not less than 2 mg and not more than 15 mg of siponimod.
Embodiment 2.11: Siponimod for use in the treatment of stroke in a human subject to any of the embodiments 2.1 to 2.10, wherein the maintenance daily dose of step (b)(i) is not less than 2 mg and not more than 10 mg of siponimod.
Embodiment 2.12: Siponimod for use in the treatment of stroke in a human subject according to any of the embodiments 2.1 to 2.11, wherein the maintenance daily dose of step (b)(i) is not less than 2 mg and not more than 5 mg of siponimod.
Embodiment 2.13: Siponimod for use in the treatment of stroke in a human subject according to any of the embodiments 2.1 to 2.9, wherein the maintenance daily dose of step (b)(i) is 20 mg of siponimod.
Embodiment 2.14: Siponimod for use in the treatment of stroke in a human subject according to any of the embodiments 2.1 to 2.10, wherein the maintenance daily dose of step (b)(i) is 15 mg of siponimod.
Embodiment 2.15: Siponimod for use in the treatment of stroke in a human subject according to any of the embodiments 2.1 to 2.11, wherein the maintenance daily dose of step (b)(i) is 10 mg of siponimod.
Embodiment 2.16: Siponimod for use in the treatment of stroke in a human subject according to any of the embodiments 2.1 to 2.12, wherein the maintenance daily dose of step (b)(i) is 5 mg of siponimod.
Embodiment 2.17: Siponimod for use in the treatment of stroke in a human subject according to any of the embodiments 2.1 to 2.12, wherein the maintenance daily dose of step (b)(i) is 2 mg of siponimod.
Embodiment 2.18: Siponimod for use in the treatment of stroke in a human subject according to any of the embodiments 2.1 to 2.17, wherein the maintenance daily dose of siponimod administered in step (b) is administered for a maintenance time period of at least 3 days, e.g. for a maintenance time period of 3 or 4 days.
Embodiment 2.19: Siponimod for use in the treatment of stroke in a human subject according to any of the embodiments 2.1 to 2.18, wherein the maintenance daily dose of siponimod administered in step (b) is administered for a maintenance time period of at least 5 days, e.g. for a maintenance time period of 5 days.
Embodiment 2.20: Siponimod for use in the treatment of stroke in a human subject according to any of the embodiments 2.1 to 2.19, wherein the maintenance daily dose of siponimod administered in step (b) is administered for a maintenance time period of at least 7 days, e.g. for a maintenance time period of 12 days.
Embodiment 2.21: Siponimod for use in the treatment of stroke in a human subject according to any of the embodiments 2.1 to 2.20, wherein the maintenance daily dose of siponimod administered in step (b) is administered for a maintenance time period of at least 14 days.
Embodiment 2.22: Siponimod for use in the treatment of stroke in a human subject according to any of the embodiments 2.1 to 2.21, wherein the daily maintenance dose of siponimod administered in step (b) is administered for a maintenance time period of at least 21 days.
Embodiment 2.23: Siponimod for use in the treatment of stroke in a human subject according to any of the embodiments 2.1 to 2.22, wherein the maintenance daily dose of siponimod administered in step (b) is administered for a maintenance time period of at least 28 days.
Embodiment 2.24: Siponimod for use in the treatment of stroke in a human subject according to any of the embodiments 2.1 to 2.23, wherein the maintenance daily dose of siponimod administered in step (b) is administered for a maintenance time period of at least 35 days.
Embodiment 2.25: Siponimod for use in the treatment of stroke in a human subject according to any of the embodiments 2.1 to 2.24, the administration of the maintenance daily dose of siponimod in step (b) comprises intravenous administration.
Embodiment 2.26: Siponimod for use in the treatment of stroke in a human subject according to any of the embodiments 2.1 to 2.25, the administration of the maintenance daily dose of siponimod in step (b) comprises oral administration.
Embodiment 2.27: Siponimod for use in the treatment of stroke in a human subject according to any of the embodiments 2.1 to 2.25, wherein the maintenance daily dose of siponimod in step (b) is administered in a first phase intravenously and in a second phase orally, wherein preferably the first phase has a duration of 5 days and the second phase has a duration of 7 days.
Embodiment 2.28: Siponimod for use in the treatment of stroke in a human subject according to any of the embodiments 2.1 to 2.27, wherein said subject is continuously monitoring said subject via cardiovascular telemetry for at least the first 24 hours, preferably for at least the first 48 hours calculated starting from the administration of the first dose of siponimod.
Embodiment 2.29: Siponimod for use in the treatment of stroke in a human subject according to any of the embodiments 2.1. to 2.28, wherein if a consecutive dose of siponimod in step (a) is increased by an increment, said increment is governed by a modified Fibonacci series, i.e. a given dose is the sum of two directly previous doses ±40%, for example ±35%, for example ±30%, for example ±20%, e.g. about ±23%, or for example ±10%.
Embodiment 2.30: Siponimod for use in the treatment of stroke in a human subject according to any of the embodiments 2.1, 2.3, 2.5, 2.10, 2.11, 2.15 or from 2.18 to 2.29, wherein
(a) multiple consecutive doses of siponimod are intravenously administered to said subject over a time period equal to or up to 48 hours calculated starting at the first intravenously administered dose, wherein
on Day 1, the administered doses are 0.25 mg over 6 hours, then 0.25 mg over 6 hours, then 0.5 mg over 6 hours, and then 0.75 mg over 6 hours for a total Day 1 dose of 1.75 mg; and
on Day 2, the administered doses are 1.25 mg over 6 hours, then 2 mg over 6 hours, then 2.5 mg over 6, and then 2.5 mg over 6 hours for a total Day 2 dose of 8.25 mg; and
(b) a maintenance daily dose of 10 mg of siponimod is intravenously administered on Day 3 through Day 7; and
optionally the maintenance daily dose of 10 mg of siponimod is orally administered on and after Day 8, preferably on Day 8 through Day 14; and wherein
(c) optionally said subject is continuously monitored via cardiovascular telemetry for at least the first 24 hours, preferably for at least the first 48 hours, calculated starting from the administration of the first dose of siponimod.
Embodiment 2.31: Siponimod for use in the treatment of stroke in a human subject according to any of the embodiments 2.1 to 2.30, wherein when in step (b) siponimod is orally administered it is administered in the form of an oral solid dosage form.
Embodiment 2.32: Siponimod for use in the treatment of stroke in a human subject according to the embodiment 2.31, wherein the oral solid dosage form of step (b) is an immediate release oral solid dosage form.
Embodiment 2.33: Siponimod for use in the treatment of stroke in a human subject according to the embodiment 2.32, wherein the oral immediate release solid dosage form administered in step
(b) is in the form of tablets having the composition as provided in Table 2.1 or Table 2.2.
Embodiment 2.34: Siponimod for use in the treatment of stroke in a human subject suffering from stroke according to any of the embodiments 2.31 to 2.33, wherein the 10 mg maintenance daily dose of siponimod of step (b) is administered to said subject in the form of
   (a) 5 tablets of 2 mg strength; or
   (b) 2 tablets of 5 mg strength; or
   (c) 1 tablet of 10 mg strength;
and wherein when the 10 mg daily dose of siponimod is administered by more than 1 tablet, the tablets are administered simultaneously, sequentially or separately, preferably simultaneously.
Embodiment 2.35: Siponimod for use in the treatment of stroke in a human subject suffering from stroke, preferably hemorrhagic stroke, more preferably ICH, as defined in any of the preceding embodiments 2.1 to 2.34, wherein the administered i.v. composition containing siponimod is obtained by diluting, for example in saline or 5% glucose solution, a concentrate containing siponimod, wherein the concentrate
   (i) is in the form of a liquid;
   (ii) contains 1 mg/mL of siponimod; and
   (iii) contains
      7 wt. %-13 wt. %of 2-hydroxypropyl-f3-cyclodextrin (HPBCD);
      a buffer agent; and
      optionally a tonicity agent.

Embodiment 2.36: Siponimod for use in the treatment of stroke in a human subject suffering from stroke, as defined in the embodiment 2.35, wherein stroke is preferably hemorrhagic stroke, more preferably ICH and wherein the administered i.v. composition containing siponimod is obtained by diluting, for example in saline or 5% glucose solution, a concentrate containing siponimod, wherein the concentrate
(i) is in the form of a liquid; and
(ii) contains 1 mg/mL of siponimod; and in addition
(iii) contains
   10 wt. % of 2-hydroxypropyl-f3-cyclodextrin (HPBCD);
   3 wt. % mannitol; and
   0.06 wt. % 2-amino-2-(hydroxymethyl)propan-1,3-diol (Tris); and
(iv) its pH is about 8.
Embodiments 2.37: Siponimod for use in the treatment of stroke in a human subject as defined in any of the preceding embodiments 2.1 to 2.36, wherein stroke is preferably hemorrhagic stroke, more preferably ICH and wherein the first dose of siponimod is administered within 72 hours, preferably within 48 hours, more preferably within 24 hours, e.g. within 6 or 12 hours, from the onset of ICH.
Embodiment 2.38: Siponimod for use in the treatment of stroke in a human subject as defined in any of the preceding embodiments 2.1 to 2.37, wherein stroke is intracerebral hemorrhagic stroke (ICH).
Embodiment 2.39: Siponimod for use in the treatment of stroke in a human subject as defined in any of the preceding embodiments 2.1 to 2.38, wherein stroke, e.g. ICH, is grade 4 stroke or higher as defined by the National Institute of Health Stroke Scale (NIHSS).
Embodiment 2.40: Siponimod for use in the treatment of stroke in a human subject suffering from stroke as defined in any of the preceding embodiments 2.1. to 2.39, wherein stroke, e.g. ICH, is grade 6 stroke or lower as defined by the National Institute of Health Stroke Scale (NIHSS).
Embodiments 2.41: Siponimod for use in the treatment of stroke in a human subject suffering from stroke as defined in any of the preceding embodiments 2.1 to 2.40, wherein stroke is a spontaneous intracerebral haemorrhage (SICH), wherein the supratentorial intracerebral hemorrhage is in deep brain structures (putamen, thalamus, caudate, and associated deep white matter tracts) with a volume 10 mL but 30 mL calculated by the ABC/2 method) determined by routine clinical magnetic resonance imaging (MRI) or computerized tomography (CT).
Embodiments 2.42: Siponimod for use in the treatment of stroke, e.g. ICH, in a human subject as defined in any of the preceding embodiments 2.1 to 2.41, wherein said subject has a Glasgow Coma Scale (GCS) motor score which is no less than 6.
Embodiment 2.43: Siponimod for use in the treatment of stroke, e.g. ICH, in a human subject as defined in any of the preceding embodiments 2.1 to 2.42, wherein said subject is a CYP2C9*2*3 poor metabolizer or a CYP2C9*3*3 poor metabolizer.
Embodiment 2.44: Siponimod for use in the treatment of stroke, e.g. ICH, in a human subject as defined in any of the preceding embodiments 2.1 to 2.43, wherein siponimod is contained in an oral solid dosage form and is in the form of a co-crystal with fumaric acid.
Embodiment 2.45: Siponimod for use in the improvement of the global functioning of human subject suffering from stroke, in particular from ICH, wherein the improvement is measured by the modified Rankin Scale (mRS) on Day 90 after ICH to achieve a mRS score equal to 0, 1 or 2 and wherein siponimod is administered according to the use for treatment as defined in any of the preceding embodiments 2.1 to 2.44.
Embodiment 3.1: Use of siponimod or pharmaceutically acceptable salts, co-crystals, hydrates, solvates, polymorphs of siponimod and/or mixtures thereof for the manufacture of a medicament for the treatment of stroke in a human subject suffering from stroke, wherein said use comprises
(a) intravenously administering multiple consecutive doses of siponimod to said subject over a time period equal to or up to 96 hours calculated starting at the first intravenously administered dose, wherein
(i) the first administered dose is not less than 0.25 mg and not more than 1.25 mg; and wherein
(ii) each dose of the one or more consecutive doses administered after the first dose is not less than the directly preceding administered dose and not more than the directly subsequent administered dose; and wherein
(iii) the total sum of the consecutive doses administered over a time period of 24 consecutive hours is lower than the maintenance daily dose of siponimod; and wherein said use further comprises subsequently
(b) administering the maintenance daily dose of siponimod for a maintenance time period of at least 2 days, wherein
(i) the daily maintenance dose is not less than 2 mg and not more than 20 mg of siponimod.
Embodiment 3.2: Use of siponimod or pharmaceutically acceptable salts, co-crystals, hydrates, solvates, polymorphs of siponimod and/or mixtures thereof for the manufacture of a medicament for the treatment of stroke in a human subject according to the embodiment 3.1, wherein the multiple consecutive doses of siponimod are intravenously administrated to said subject according to step (a) over a time period equal to or up to 72 hours calculated starting at the first intravenously administered dose.
Embodiment 3.3: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 or 3.2, wherein the multiple consecutive doses of siponimod are intravenously administrated to said subject according to step (a) over a time period equal to or up to 48 hours calculated starting at the first intravenously administered dose.
Embodiment 3.4: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.3, wherein multiple consecutive doses of siponimod are intravenously administrated to said subject according to step (a) over a time period equal to or up to 48 hours calculated starting at the first intravenously administered dose.
Embodiment 3.5: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.4, wherein the first administered dose of step (a) is 0.25 mg of siponimod.
Embodiment 3.6: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.4, wherein the first administered dose of step (a) is 0.5 mg of siponimod.
Embodiment 3.7: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.4, wherein the first administered dose of step (a) is 0.75 mg of siponimod.
Embodiment 3.8: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.4, wherein the first administered dose of step (a) is 1.0 mg of siponimod.
Embodiment 3.9: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.4, wherein the first administered dose of step (a) is 1.25 mg of siponimod.
Embodiment 3.10: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.9, wherein the maintenance daily dose of step (b)(i) is not less than 2 mg and not more than 15 mg of siponimod.
Embodiment 3.11: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.10, wherein the maintenance daily dose of step (b)(i) is not less than 2 mg and not more than 10 mg of siponimod.
Embodiment 3.12: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.11, wherein the maintenance daily dose of step (b)(i) is not less than 2 mg and not more than 5 mg of siponimod.
Embodiment 3.13: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.9, wherein the maintenance daily dose of step (b)(i) is 20 mg of siponimod.
Embodiment 3.14: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.10, wherein the maintenance daily dose of step (b)(i) is 15 mg of siponimod.
Embodiment 3.15: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.11, wherein the maintenance daily dose of step (b)(i) is 10 mg of siponimod.
Embodiment 3.16: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.12, wherein the maintenance daily dose of step (b)(i) is 5 mg of siponimod.
Embodiment 3.17: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.12, wherein the maintenance daily dose of step (b)(i) is 2 mg of siponimod.
Embodiment 3.18: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.17, wherein the maintenance daily dose of siponimod administered in step (b) is administered for a maintenance time period of at least 3 days, e.g. for a maintenance time period of 3 or 4 days.
Embodiment 3.19: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.18, wherein the maintenance daily dose of siponimod administered in step (b) is administered for a maintenance time period of at least 5 days, e.g. for a maintenance time period of 5 days.
Embodiment 3.20: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.19, wherein the maintenance daily dose of siponimod administered in step (b) is administered for a maintenance time period of at least 7 days, e.g. for a maintenance time period of 12 days.
Embodiment 3.21: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.20, wherein the maintenance daily dose of siponimod administered in step (b) is administered for a maintenance time period of at least 14 days.
Embodiment 3.22: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.21, wherein the daily maintenance dose of siponimod administered in step (b) is administered for a maintenance time period of at least 21 days.
Embodiment 3.23: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.22, wherein the maintenance daily dose of siponimod administered in step (b) is administered for a maintenance time period of at least 28 days.
Embodiment 3.24: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.23, wherein the maintenance daily dose of siponimod administered in step (b) is administered for a maintenance time period of at least 35 days.
Embodiment 3.25: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.24, wherein the administration of the maintenance daily dose of siponimod in step (b) comprises intravenous administration.
Embodiment 3.26: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.25, wherein the administration of the maintenance daily dose of siponimod in step (b) comprises oral administration.
Embodiment 3.27: Use of siponimod or pharmaceutically acceptable salts, co-crystals, hydrates, solvates, polymorphs of siponimod and/or mixtures thereof for the manufacture of a medicament for the treatment of stroke in human a subject according to any of the embodiments 3.1 to 3.26, wherein the maintenance daily dose of siponimod in step (b) is administered in a first phase intravenously and in a second phase orally, wherein preferably the first phase has a duration of 5 days and the second phase has a duration of 7 days.
Embodiment 3.28: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.27, wherein said use further comprises
(c) continuously monitoring said subject via cardiovascular telemetry for at least the first 24 hours, preferably for at least the first 48 hours calculated starting from the administration of the first dose of siponimod.
Embodiment 3.29: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1. to 3.28, wherein if a consecutive dose of siponimod in step (a) is increased by an increment, said increment is governed by a modified Fibonacci series, i.e. a given dose is the sum of two directly previous doses ±40%, for example ±35%, for example ±30%, for example ±20%, e.g. about ±23%, or for example ±10%.
Embodiment 3.30: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 1.3, 3.3, 3.5, 3.10, 3.11, 3.15 or from 3.18 to 3.29, wherein said use comprises
(a) intravenously administering multiple consecutive doses of siponimod to said subject over a time period equal to or up to 48 hours calculated starting at the first intravenously administered dose, wherein
on Day 1, the administered doses are 0.25 mg over 6 hours, then 0.25 mg over 6 hours, then 0.5 mg over 6 hours, and then 0.75 mg over 6 hours for a total Day 1 dose of 1.75 mg; and
on Day 2, the administered doses are 1.25 mg over 6 hours, then 2 mg over 6 hours, then 2.5 mg over 6, and then 2.5 mg over 6 hours for a total Day 2 dose of 8.25 mg; and
(b) intravenously administering a maintenance daily dose of 10 mg of siponimod is intravenously administered on Day 3 through Day 7; and
optionally orally administering the maintenance daily dose of 10 mg of siponimod on and after Day 8, preferably on Day 8 through Day 14; and wherein said treatment optionally further comprises
(c) continuously monitoring said subject via cardiovascular telemetry for at least the first 24 hours, preferably for at least the first 48 hours, calculated starting from the administration of the first dose of siponimod.
Embodiment 3.31: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.1 to 3.30, wherein when in step (b) siponimod is administered orally it is administered in the form of an oral solid dosage form.
Embodiment 3.32: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.31, wherein the oral solid dosage form of step (b) is an immediate release oral solid dosage form.
Embodiment 3.33: Use of siponimod or pharmaceutically acceptable salts, co-crystals, hydrates, solvates, polymorphs of siponimod and/or mixtures thereof for the manufacture of a medicament for the treatment of stroke in a human subject according to the embodiment 3.32, wherein the oral immediate release solid dosage form administered in step (b) is in the form of tablets having the composition as provided in Table 2.1 or Table 2.2.
Embodiment 3.34: Use of siponimod or pharmaceutically acceptable salts, co-crystals, hydrates, solvates, polymorphs of siponimod and/or mixtures thereof for the manufacture of a medicament for the treatment of stroke in a human subject according to any of the embodiments 3.31 to 3.33, wherein the 10 mg maintenance daily dose of siponimod of step (b) is administered to the human subject in need thereof in the form of
   (a) 5 tablets of 2 mg strength; or
   (b) 2 tablets of 5 mg strength; or
   (c) 1 tablet of 10 mg strength;
and wherein when the 10 mg daily dose of siponimod is administered by more than 1 tablet, the tablets are administered simultaneously, sequentially or separately, preferably simultaneously.
Embodiment 3.35: Use of siponimod or pharmaceutically acceptable salts, co-crystals, hydrates, solvates, polymorphs of siponimod and/or mixtures thereof for the manufacture of a medicament for the treatment of stroke, preferably hemorrhagic stroke, more preferably ICH, in a human subject, as defined in any of the preceding embodiments 3.1. to 3.34, wherein the administered i.v. composition containing siponimod is directly obtained by diluting, for example in saline or 5% glucose solution, a concentrate containing siponimod, wherein the concentrate
   (i) is in the form of a liquid;
   (ii) contains 1 mg/mL of siponimod; and
   (iii) contains
      7 wt. %-13 wt. % of 2-hydroxypropyl-f3-cyclodextrin (HPBCD);
      a buffer agent; and
      optionally a tonicity agent.
Embodiment 3.36: Use of siponimod or pharmaceutically acceptable salts, co-crystals, hydrates, solvates, polymorphs of siponimod and/or mixtures thereof for the manufacture of a medicament for the treatment of stroke in a human subject according to the embodiment 3.35, wherein stroke is preferably hemorrhagic stroke, more preferably ICH and wherein the administered i.v. composition containing siponimod is directly obtained by diluting, for example in saline or 5% glucose solution, a concentrate containing siponimod and wherein the concentrate
   (i) is in the form of a liquid; and
   (ii) contains 1 mg/mL of siponimod; and in addition
   (iii) contains
      10 wt. % of 2-hydroxypropyl-f3-cyclodextrin (HPBCD);
      3 wt. % mannitol; and
      0.06 wt. % 2-amino-2-(hydroxymethyl)propan-1,3-diol (Tris); and
   (iv) its pH is about 8.

Embodiments 3.37: Use of siponimod or pharmaceutically acceptable salts, co-crystals, hydrates, solvates, polymorphs of siponimod and/or mixtures thereof for the manufacture of a medicament for the treatment of stroke in a human subject according to any of the preceding embodiments 3.1 to 3.36, wherein stroke is preferably hemorrhagic stroke, more preferably ICH and wherein the first dose of siponimod of said treatment is administered within 72 hours, preferably within 48 hours, more preferably within 24 hours, e.g. within 6 or 12 hours, from the onset of ICH.
Embodiment 3.38: Use of siponimod or pharmaceutically acceptable salts, co-crystals, hydrates, solvates, polymorphs of siponimod and/or mixtures thereof for the manufacture of a medicament for the treatment of stroke in a human subject according to any of the preceding embodiments 3.1 to 3.37, wherein stroke is intracerebral hemorrhagic stroke (ICH).
Embodiment 3.39: Use of siponimod or pharmaceutically acceptable salts, co-crystals, hydrates, solvates, polymorphs of siponimod and/or mixtures thereof for the manufacture of a medicament for the treatment of stroke in a human subject as defined in any of the preceding embodiments 3.1 to 3.38, wherein stroke, e.g. ICH, is grade 4 stroke or higher as defined by the National Institute of Health Stroke Scale (NIHSS).
Embodiment 3.40: Use of siponimod or pharmaceutically acceptable salts, co-crystals, hydrates, solvates, polymorphs of siponimod and/or mixtures thereof for the manufacture of a medicament for the treatment of stroke in a human subject as defined in any of the preceding embodiments 3.1 to 3.39, wherein stroke, e.g. ICH, is grade 6 stroke or lower as defined by the National Institute of Health Stroke Scale (NIHSS).
Embodiments 3.41: Use of siponimod or pharmaceutically acceptable salts, co-crystals, hydrates, solvates, polymorphs of siponimod and/or mixtures thereof for the manufacture of a medicament for the treatment of stroke, e.g. ICH, in a human subject as defined in any of the preceding embodiments 3.1 to 3.40, wherein stroke is a spontaneous intracerebral haemorrhage (SICH), and the supratentorial intracerebral hemorrhage is in deep brain structures (putamen, thalamus, caudate, and associated deep white matter tracts) with a volume 10 mL but 30 mL calculated by the ABC/2 method) determined by routine clinical magnetic resonance imaging (MRI) or computerized tomography (CT).
Embodiments 3.42: Use of siponimod or pharmaceutically acceptable salts, co-crystals, hydrates, solvates, polymorphs of siponimod and/or mixtures thereof for the manufacture of a medicament for the treatment of stroke, e.g. ICH, in a human subject as defined in any of the preceding embodiments 3.1 to 3.41, wherein said subject has a Glasgow Coma Scale (GCS) motor score which is no less than 6.
Embodiment 3.43: Use of siponimod or pharmaceutically acceptable salts, co-crystals, hydrates, solvates, polymorphs of siponimod and/or mixtures thereof for the manufacture of a medicament for the treatment of stroke, e.g. ICH, in a human subject as defined in any of the preceding embodiments 3.1 to 3.42, wherein said subject is a CYP2C9*2*3 poor metabolizer or a CYP2C9*3*3 poor metabolizer.
Embodiment 3.44: Use of siponimod or pharmaceutically acceptable salts, co-crystals, hydrates, solvates, polymorphs of siponimod and/or mixtures thereof for the manufacture of a medicament for the treatment of stroke, e.g. ICH, in a human subject as defined in any of the preceding embodiments 3.1 to 3.43, wherein siponimod is contained in an oral solid dosage form and is in the form of a co-crystal with fumaric acid.
Embodiment 3.45: Use of siponimod or pharmaceutically acceptable salts, co-crystals, hydrates, solvates, polymorphs of siponimod and/or mixtures thereof for the manufacture of a medicament for the improvement of the global functioning of human subject suffering from stroke, in particular from ICH, wherein the improvement is measured by the modified Rankin Scale (mRS) on Day 90 after ICH to achieve a mRS score equal to 0, 1 or 2 and wherein siponimod is administered according to the use for treatment as defined in the preceding embodiments 3.1 to 3.44.

According to the disclosure, the period of treatment of step (a) refers to the period during which siponimod is administered at a daily dose lower than the maintenance daily dose. The period of treatment of step (a) starts with the first administration (e.g., the administration of the first dose) of siponimod.

The first administered dose of siponimod of the present disclosure is not less than 0.25 mg and not more than 1.25 mg. In one embodiment the first administered dose is not less than 0.25 mg and not more than 0.75 mg, e.g. preferably 0.5 mg, more preferably 0.25 mg. In another embodiment the first administered dose is between 0.75 mg and 1.25 mg, e.g. 0.75 mg or 1.0 mg, preferably 0.75 mg.

The maintenance daily dose of siponimod of step (b) of the present disclosure is not less than 2 mg and not more than 20 mg of siponimod. In one embodiment, the maintenance daily dose is not less than 2 mg and not more than 10 mg, e.g. 2 mg or 5 mg. In another embodiment, the maintenance daily dose is between 10 mg and 20 mg, e.g. 10 mg or 15 mg, preferably 10 mg. The term “daily” indicates a time period of 24 hours.

In step (a) of the method of treatment of the present disclosure the consecutive doses of siponimod are intravenously administering to a human subject suffering from stroke, preferably hemorrhagic stroke, more preferably ICH, over a time period equal to or up to 96 hours. In one embodiment the time period is between 78 and 96 hours, e.g. 84 hours or 90 hours. In another embodiment it is between 60 and 78 hours, e.g. 66 hours or 72 hours. In another embodiment it is up to 72 hours, e.g. between 42 hours and 60 hours, e.g. 48 hours or 54 hours. In another embodiment it is up to 48 hours, e.g. between 36 hours and 48 hours, e.g. 42 hours or 36 hours.

In another embodiment it is up to 40 hours e.g. between 30 and 40 hours, e.g. 33 hours or 39 hours. In another embodiment it is up to 36 hours, e.g. between 18 hours and 36 hours, e.g. 24 hours or 30 hours. In another embodiment is up to 24 hours, e.g. between 3 hours and 24 hours, e.g. 6 hours or 12 hours. In one embodiment, it is 48 hours. In one embodiment, it is 24 hours. In one embodiment, the period of treatment of step (a) terminates at the beginning of the first day in which the total dose of siponimod administered in this entire day, i.e. in its span of 24 hours, is equal to the maintenance daily dose.

In one embodiment each dose of the consecutive doses of siponimod is administered every 24 hours. In another embodiment it is administered every 12 hours. In a further embodiment it is administered every 6 hours or every 3 hours. Preferably it is administered every 6 hours. In one embodiment the maintenance daily dose of siponimod is administered for a period which is up to 90 days, for example up to 77 days, e.g. up to 63. In another embodiment it is up to 56 days, e.g. between 35 days and 56 days, for example 42 days or 49 days. In another embodiment it is administered for a period which is up to 30 days, e.g. from 25 to 30 days, for example 29 days or 28 days. Alternatively, for a period which is up to 25 days, e.g. from 20 to 25 days, for example 21 days or 24 days. Alternatively, for a period which is up to 20 days, e.g. from 15 to 20 days, for example 18 days or 19 days. Alternatively, for a period which is in the range from 10 to 14 days, e.g. 12 days or 14 days. Alternatively, for a period which may be shorter, e.g. in the range from 5 to 10 days, such as 7 or 10 days. Alternatively, siponimod may be administered in a daily dose of 10 mg for a period which may be shorter, e.g. in the range from 1 to 4 days, e.g. 1 to 3 days, such as 2 or 3 days. Preferably siponimod is administered in a daily dose of 10 mg for a period which is at least 12 days, e.g., 12 days

In one embodiment wherein the maintenance daily dose is between 2 mg and 10 mg, in step (a) of the method of treatment described in the present disclosure the administered daily dose of siponimod may be up to 9.5 mg, e.g. up to 9 mg, or up to 8.5 mg, e.g. about 8.25 mg or about 8 mg. Alternatively it may be up to 7.75 mg, e.g. about 7.5 or about 7.25 mg, or up to 7 mg, e.g. up to 6.5 mg, e.g. 6.25 mg, or up to 6 mg, e.g. up to 5.75 mg, e.g. about 5.5 mg or about 5 mg. Alternatively the daily dose of siponimod administered in step (a) of the present method of treatment may be up to 4 mg, e.g. about 3.75 mg or about 3.5 mg, or up to 3 mg, e.g. about 2.75 mg, or up to 2.5 mg, e.g. about 2.25 mg. Alternatively it may be up to 2 mg, e.g. about 1.75, or up to 1.5 mg, e.g. about 1.25 mg, or up to 1 mg, e.g. about 0.75 mg or 0.5 mg. The administered daily dose of siponimod of step (a) is lower than the maintenance daily dose of step (b).

In a further embodiment wherein the maintenance daily dose is between 10 mg and 20 mg, in step (a) of the method of treatment described in the present disclosure, the administered daily dose of siponimod may up to 19.5 mg, e.g. up to 19 mg, or up to 18.5 mg, e.g. about 18.25 mg or about 18 mg. Alternatively it may be up to 17.75 mg, e.g. about 17.5 or about 17.25 mg, or up to 17 mg, e.g. up to 16.5 mg, e.g. 16.25 mg, or up to 16 mg, e.g. up to 15.75 mg, e.g. about 15.5 mg or about 15 mg. Alternatively the daily dose of siponimod administered in step (a) of the present method of treatment may be up to 14 mg, e.g. about 13.75 mg or about 13.5 mg, or up to 13 mg, e.g. about 12.75 mg, or up to 12.5 mg, e.g. about 12.25 mg. Alternatively it may be up to 12 mg, e.g. about 11.75, or up to 11.5 mg, e.g. about 11.25 mg, or up to 11 mg, e.g. about 10.75 mg or 10.5 mg. Alternatively it may be up to 10.25, e.g. about 10 mg or about 9.75 mg, or up to 9 mg, or up to 8.5 mg, e.g. about 8.25 mg or about 8 mg. Alternatively it may be up to 7.75 mg, e.g. about 7.5 or about 7.25 mg, or up to 7 mg, e.g. up to 6.5 mg, e.g. 6.25 mg, or up to 6 mg, e.g. up to 5.75 mg, e.g. about 5.5 mg or about 5 mg. Alternatively the daily dose of siponimod administered in step (a) of the present method of treatment may be up to 4 mg, e.g. about 3.75 mg or about 3.5 mg, or up to 3 mg, e.g. about 2.75 mg, or up to 2.5 mg, e.g. about 2.25 mg. Alternatively it may be up to 2 mg, e.g. about 1.75, or up to 1.5 mg, e.g. about 1.25 mg, or up to 1 mg, e.g. about 0.75 mg or 0.5 mg. The administered daily dose of siponimod of step (a) is lower than the maintenance daily dose of step (b).

In a further embodiment, in step (a) as the daily dose administered on Day 1 of the treatment, siponimod may be administered at a dose up to 4 mg, e.g. about 3.75 mg or 3.5 mg, or up to 3 mg, e.g. up to 2.75 mg, e.g. 2.5 mg or 2.25 mg. Alternatively, in step (a), as the daily dose administered on Day 1 of the treatment, siponimod may be administered at a dose up to 2 mg, e.g. about 1.75 mg or 1.5 mg, or up to 1.25 mg, e.g. about 1 mg. Alternatively at a dose up to 0.75 mg, e.g. 0.5 mg or 0.25 mg. The administered daily dose of siponimod of step (a) is lower than the maintenance daily dose of step (b).

In a further embodiment, in step (a), as the daily dose administered on Day 2 of the treatment, siponimod may be administered at a dose up to 9 mg, e.g. about 8.75 mg or about 8.5 mg, or up to 8 mg, e.g. up to 7.75 mg, e.g. 7.5 mg or 7 mg. Alternatively, in step (a), as the daily dose administered on Day 2 of the treatment, siponimod may be administered at a dose up to 6.75 mg, e.g. about 6.5 mg or 6.25 mg, or up to 5.75 mg, e.g. about 5.5 mg or 5.25 mg. Alternatively at a dose up to 4.75 mg, e.g. about 4.5 mg or 4.25 mg. Alternatively, at a dose up to 3.75 mg, e.g. about 3.5 mg or 3.25 mg. The administered daily dose of siponimod of step (a) is lower than the maintenance daily dose of step (b).

According to the present disclosure each dose of the one or more consecutive doses administered after the first dose in step (a) is: (a)(ii) not less than the directly preceding administered dose and not more than the directly subsequent administered dose and (a)(iii) the total sum of the consecutive doses administered over a time period of 24 consecutive hours is lower than the maintenance daily dose.

Under the above conditions (a)(ii) and (a)(iii) of the above paragraph, in an embodiment wherein the maintenance daily dose of siponimod is 2 mg, the dose of siponimod administered in step (a) of the method of treatment may, on any given administration, be about 8-fold smaller, or about 4-fold smaller, or about between 8-fold smaller and 4-fold smaller, or about 3-fold smaller, e.g. 2.7-fold smaller or about 2-fold smaller, e.g. 1.6-fold smaller than 2 mg of siponimod.

Under the above conditions (a)(ii) and (a)(iii) of the above paragraph, in an embodiment wherein the maintenance daily dose of siponimod is 5 mg, the dose of siponimod administered in step (a) of the method of treatment may, on any given administration, be about 20-fold smaller, or about 10-fold smaller, or about between 8-fold smaller and 5-fold smaller, e.g. about 6.7-fold smaller, or about 4-fold smaller, about 3-fold smaller, e.g. about 3.3-fold smaller or 2.7-fold smaller, or about 2-fold smaller than 5 mg of siponimod.

Under the above conditions (a)(ii) and (a)(iii) of the above paragraph, in an embodiment wherein the maintenance daily dose of siponimod is 10 mg, the dose of siponimod administered in step (a) of the method of treatment may, on any given administration, be about 40-fold smaller, or about 20-fold smaller, or about 15-fold smaller, e.g. about 13.3-fold smaller, or about 10-fold smaller, about 8-fold smaller, or about 6.7-fold smaller or 5-fold, e.g. about 4-fold smaller than 10 mg of siponimod.

Under the above conditions (a)(ii) and (a)(iii) of the above paragraph, in an embodiment wherein the maintenance daily dose of siponimod is 20 mg, the dose of siponimod administered in step (a) of the method of treatment may, on any given administration, be about 80-fold smaller, or about 40-fold smaller, or about 30-fold smaller, e.g. about 27-fold smaller, or about 15-fold smaller, e.g. 13-fold smaller, or about 8-fold smaller, smaller than 20 mg of siponimod.

In a further embodiment under the proviso that the sum of the doses administered in one day, i.e. in a time span of 24 hours, in step (a) of the method of the present disclosure is lower than the maintenance daily dose of step (b) and is increased stepwise in a defined incremental ratio up to the maintenance daily dose of siponimod, preferably, the administered dose of siponimod during the initial 7 days of treatment, e.g. from Day 1 to Day 7, or preferably during the initial 6 days, e.g. from Day 1 to Day 6, or preferably during the initial 5 days, e.g. from Day 1 to Day 5, or preferably during the initial 4 days, e.g. from Day 1 to Day 4, or more preferably during the initial 3 days, e.g. from Day 1 to Day 3, or even more preferably during the initial 2 days, e.g. from Day 1 to Day 2, is increased stepwise at each administration and each administered dose is from 0.1-fold up to 3-fold higher than the directly previous dose of siponimod, for example from 0.1-fold up to 2.5-fold higher, or preferably from 0.1-fold up to 2-fold higher, for example from 0.2-fold to 1.7-fold higher, e.g. from 0.2-fold up to 1.5-fold higher, e.g. 0.5-fold or 1-fold higher than the directly previous dose of siponimod.

In one embodiment, the number of consecutive doses administered in step (a) of the method of treatment of the present disclosure may be up to 32, e.g. between 20 and 32, e.g. 26 or 28. It may be further be up to 24, e.g. between 20 and 24, e.g. 18 or 16. It may be alternatively be up to 18, e.g. between 10 and 18, e.g. 12 or 14. It may be further be up to 12, e.g. between 6 and 12, e.g. 10 and 8. Alternatively it may be up to 6, e.g. between 2 and 5, e.g. 3 or 4.

### Siponimod

The IUPAC name of siponimod is 1-{4-[(1E)-N-{[4-cyclohexyl-3-(trifluoromethyl)benzyl]oxy}ethanimidoyl]-2-ethylbenzyl}-3-azetidine carboxylic acid and the compound is represented by the chemical structure according to Formula (I):,

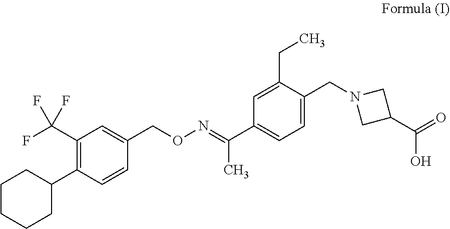 [SMILES: CCc1cc(/C(C)=N/OCc2ccc(C3CCCCC3)c(C(F)(F)F)c2)ccc1CN1CC(C(=O)O)C1]

Siponimod is a selective sphingosine-1-phosphate receptor modulator which is used in the treatment of autoimmune diseases, such multiple sclerosis (MS) and in the treatment of neurodegenerative diseases.

WO 2004/103306 A2 relates to immunosuppressant compounds and processes for their production. Inter alia, a synthesis pathway for siponimod is described. In WO 2013/113915 Al an alternative synthesis pathway for siponimod is described. Further, WO 2004/103306 A2 mentions that siponimod can generally be administered by any conventional administration route such as enterally, parentally, topically and in nasal or suppository form. However, said document does not describe any specific dose form.

Sphingosine-1-phosphate (S1P) receptors belong to a family of closely related, lipid-activated G-protein-coupled receptors. S1P1, S1P2, S1P3, S1P4, and S1P5 (also respectively termed EDG-1, EDG-5, EDG-3, EDG-6 and EDG-8) are identified as receptors specific for S1P. Certain S1P receptors are associated with diseases mediated by lymphocyte interactions, for example, in transplantation rejections, autoimmune diseases, e.g. MS and inflammatory myopathies, inflammatory diseases, infectious diseases and cancer.

Siponimod selectively targets S1P receptor subtypes 1 and 5. It is currently in Phase 3 EXPAND clinical development as an oral formulation for the treatment of multiple sclerosis (MS), specifically secondary progressive MS (SPMS). The use of siponimod as a medicament in stroke was generically first mentioned in WO 2010/080409 Al, WO 2010/080455 Al, WO 2010/071794 A1 and WO 2012/093161. Said documents however do not provide any guidance as to its specific use in stroke, or any method of treatment for a patient suffering from stroke and any specific dosage form suitable for parenteral administration.

Siponimod acts as a selective modulator of two of the five sphingosine-1-phosphate (S1P) receptors: S1P1 and S1P5. T cells selectively require S1P1 activation for emigration from the thymus, and both T- and B cells require this receptor for egress from peripheral lymphoid organs (Matloubian et al. 2004, Brinkmann et al. 2004). Pre-clinical data from mice with defective expression of S1P1 in lymphocytes propose an obligatory role of S1P1 in the egress of lymphocytes from lymphatic tissues.

Siponimod is a second generation S1P receptor modulator that reduces peripheral lymphocyte counts approximately 4-6 hours (h) after the first dose. The half-life of Siponimod is approximately 30 hours, which allows reversal of pharmacodynamic effects and recovery of the baseline lymphocyte counts within a week after treatment withdrawal. Siponimod's mode of action is believed to include S1P1-mediated prevention of effector lymphocyte recirculation from lymphatic tissue to sites of inflammation, such as the central nervous system (CNS). In addition, there may be direct beneficial effects in the CNS mediated by S1P1 and/or S1P5. Siponimod readily crosses the blood brain barrier and evidence from preclinical models suggests that siponimod may target S1P1 and S1P5 on neurons, astrocytes and oligodendrocytes and may modulate neurobiological processes (Choi et al 2011). Thus, siponimod may display additional beneficial activities in the CNS.

The dosing regimen of the present disclosure reduces peripheral leukocyte count acutely after ICH and in this way decreases secondary injury after ICH and thereby to improve outcomes.

The pharmaceutical compositions used in the treatment of stroke may contain siponimod as a free form or as pharmaceutically acceptable salts, hydrates, solvates, polymorphs, co-crystals and/or mixtures thereof. In a preferred embodiment siponimod is added to the formulation in form of an acid addition product, such as a salt or a co-crystal. In a more preferred embodiment, siponimod is added as a pharmaceutically acceptable co-crystal.

The pharmaceutically acceptable salts can e.g. be obtained by the reaction of siponimod with an acid. Examples of pharmaceutically acceptable salts of the compound of siponimod include salts with inorganic acids, such as hydrochloride, hydrobromide and sulfate, as well as salts with organic acids such as acetic acid, maleic acid, benzoic acid, citric acid, malic acid, as well as salts with sulfonic acid, such as methanesulfonic acid or benzenesulfonic acid, or, when appropriate, salts with metals, such as sodium, potassium, calcium and aluminium, salts with amines, such as trimethylamine, and salts with dibasic amino acids, such as lysine.

The compounds and salts of the combination of the pharmaceutical composition encompass hydrate and solvate forms. In a preferred pharmaceutical composition siponimod is in form of an acid addition product with fumaric acid. In a more preferred pharmaceutical composition siponimod is in form of a co-crystal.

Generally, a co-crystal can be referred to as crystalline material composed of two or more different molecules in the same lattice, wherein these two or more molecules are non-volatile. Co-crystals can be preferably be distinguished from salts because unlike salts their components are in a neutral state and interact non-ionically.

In particular preferred pharmaceutical compositions, siponimod is in the form of a co-crystal of siponimod with fumaric acid, hereinafter also referred to as (1-{4-[(1E)-N-{[4-cyclohexyl-3-(trifluoromethyl)benzyl]oxy}ethanimidoyl]-2-ethylbenzyl}-3-azetidinecarboxylic acid-fumaric acid co-crystal.

The ratio of fumaric acid, i.e. (2E)-But-2-enedioic acid, to 1-{4-[(1E)-N-{[4-cyclohexyl-3-(trifluoromethyl)benzyl]oxy}ethanimidoyl]-2-ethylbenzyl}-3-azetidinecarboxylic acid can e.g. range from 0.3 to 0.7, preferably it can be about 0.5.

The IUPAC name of the preferred co-crystal of siponimod with fumaric acid is (2E)-But-2-enedioic acid—1-({4-[(1E)-N-{[4-cyclohexyl-3(trifluoromethyl)phenyl]methoxy}ethanimidoyl]-2-ethylphenyl}methyl)azetidine-3-carboxylic acid (1:2).

In still more preferred pharmaceutical compositions, siponimod is used as 1-{4-[(1E)-N-{[4-cyclohexyl-3-(trifluoromethyl)benzyl]oxy}ethanimidoyl]-2-ethyl benzyl}-3-azetidinecarboxylic acid-fumaric acid co-crystal in polymorphic form A having an X-ray powder diffraction pattern with specific peaks at 6.9, 10.1, 10.6, 12.1, 17.5 18.1 and 20.7° (2θ).

In still more preferred pharmaceutical compositions, siponimod is used as 1-{4-[(1E)-N-{[4-cyclohexyl-3-(trifluoromethyl)benzyl]oxy}ethanimidoyl]-2-ethylbenzyl}-3-azetidinecarboxylic acid-fumaric acid co-crystal in polymorphic form A having an X-ray powder diffraction pattern (XRPD pattern) with specific peaks at 6.9, 10.1, 10.6, 12.1, 17.5 18.1 and 20.7° (2θ).

In alternatively preferred pharmaceutical compositions, siponimod is used in the free form. Unless otherwise mentioned within the present application the amounts or weight-% of siponimod are based on the amount of siponimod in free form. That is, if siponimod is present in form of a salt, the amount of the free from has to be calculated accordingly. For example, if siponimod is present in the form of its HCl salt in an amount of 1.00 g, this amount corresponds to circa 0.93 of free siponimod.

In further pharmaceutical compositions, the parenteral formulation can comprise further APIs, preferably APIs suitable to enhance the effect of the parenteral formulation. Further APIs may comprise other drugs, e.g. immunosuppressant(s), steroids(s), such as prednisolone, methylprednisolone dexamethasone, hydrocortisone and the like or nonsteroidal anti-inflammatory agent(s). The dosing regimen of a combination treatment may depend on the effectiveness and site of action of each active agent as well as synergistic effects between the agents used for combination therapy.

In alternative preferred pharmaceutical compositions, siponimod is used as the sole active pharmaceutical ingredient in the formulation and/or the treatment according to the present disclosure.

The parenteral formulation preferably contains siponimod in a concentration of 0.05 to 3.5 mg/mL, preferably 0.1 to 2.0 m/mL, more preferably 0.015 to 1.5 mg/mL. In a particularly preferred pharmaceutical composition the parenteral formulation being present in form of a concentrate can contain siponimod in concentrations of 0.25 mg/mL, 0.5 mg/mL or 1.0 mg/mL, especially 1 mg/mL. As far as the before-described concentration of siponimod is concerned, this applies to a parenteral formulation being present as a concentrate; i.e. in not further diluted form. It is evident that the concentration gets smaller, if the concentrate is further diluted for example to form an infusion solution.

### Formulations

In one embodiment the drug product comprising siponimod is a solid form, e.g., tablet, suitable for oral administration.

In a further embodiment the drug product comprising siponimod is in the form of a concentrate, e.g. liquid in vial, suitable for parenteral administration, e.g. infusion or intravenous administration (i.v. administration).

### Pharmaceutical Composition Containing Siponimod for Oral Administration

Siponimod is available as film-coated tablets for oral administration. Oral dosage forms of siponimod are known in the art. Tablets containing siponimod, for example, are described in WO 2012/093161 Al and WO 2015/155711 Al. Further, WO 2007/021666 A2 relates to oral liquids of S1P-receptor agonists.

Examples of oral solid compositions of siponimod are the film-coated tablets provided hereinafter:

TABLE 2.1
Qualitative composition of siponimod film-coated tablets

| Tablets (composition 1) | Tablets (composition 2) |
|---|---|
| Strengths: 0.1 mg, 0.25 mg, | Strengths: 0.25 mg, 0.5 mg, |
| 1 mg, 4 mg, 5 mg | 1 mg, 2 mg |
| Tablet core: | Tablet core: |
| Siponimod drug substance | Siponimod drug substance |
| Lactose monohydrate (Ph. Eur./NF) | Lactose monohydrate (Ph. Eur./NF) |
| Microcrystalline cellulose/(Ph. Eur./NF) | Microcrystalline cellulose/(Ph. Eur./NF) |
| Crospovidone (Ph. Eur./NF) | Crospovidone (Ph. Eur./NF) |
| Magnesium stearate (Ph. Eur./NF) | Glyceryl behenate (Ph. Eur./NF) |
| Silica, colloidal anhydrous/ | Silica, colloidal anhydrous/ |
| Colloidal silicon dioxide (Ph. Eur./NF) | Colloidal silicon dioxide (Ph. Eur./NF) |
| Film-coat: | Film-coat: |
| Polyvinyl alcohol-partially hydrolyzed | Polyvinyl alcohol-partially hydrolyzed |
| (Ph. Eur./USP) | (Ph. Eur./USP) |
| Titanium dioxide (Ph. Eur./USP) | Titanium dioxide (Ph. Eur./USP) |
| Talc (Ph. Eur./USP) | Talc (Ph. Eur./USP) |
| Lecithin (soya) (NF) | Lecithin (soya) (NF) |
| Xanthan gum (Ph. Eur./NF) | Xanthan gum (Ph. Eur./NF) |

The film-coated tablets are packed in high density polyethylene (HDPE) bottles with induction seals (with or without a desiccant). They may also be packaged in polyvinylchloride/polychlorotrifluoroethylene-Alu or Alu-alu blisters.

A further example of an oral solid composition in the form of a 2 mg tablet is provided hereinafter.

TABLE 2.2
Siponimod 2 mg Tablets

| | | Composition | Composition |
|---|---|---|---|
| | Ingredients | per unit [%] | per unit [mg/unit] |
| Tablet Core | | | |
| | Siponimod-fumaric acid co-crystal | 2.482 | 2.2240 |
| | Lactose (spray-dried) | 67.244 | 60.2510 |
| | Cellulose MK GR | 14.230 | 12.7500 |
| | Polyvinylpolypyrrolidon XL | 5.692 | 5.1000 |
| | Aerosil 200 | 0.474 | 0.4250 |
| | Glyceryl behenate | 4.743 | 4.2500 |
| Total core tablet | | 94.87% | 85.0000 |
| | | | mg |
| Coating | | | |
| | Coating premix white FMP.001 | 4.962 | 4.4459 |
| | Coating premix yellow FMP.001 | 0.164 | 0.1472 |
| | Coating premix red FMP.001 | 0.008 | 0.0069 |
| Total film coat (solids) | | 5.13% | 4.6000 |
| | Purified water ⁽*⁾ | — | 18.4000 |
| Total film coating tablet | | 100.00% | 89.6000 |
| | | | mg |

⁽*⁾ Removed during processing. Solution made with 20% solid concentration.

Pharmaceutical composition containing siponimod for parental administration

Generally, a parenteral formulation can be regarded as a formulation which is administered by bypassing the gastrointestinal tract. Reference is made to Ph.Eur. 8.0, section “Parenteralia”. In a preferred embodiment the formulation of the present disclosure is administered by infusion or injection. In particular, the formulation of the present disclosure is administered intravenously.

In the parenteral formulation used in the present disclosure siponimod is present in liquid form. Preferably, the parenteral formulation comprising siponimod is a solution. Suspensions are less preferred. Preferably the parenteral formulation comprising siponimod is in form of a concentrate.

Within this application a “concentrate” is referred to as a parenteral formulation which preferably is not administered directly to a patient but diluted before administration. For example, the concentrate can be diluted with a suitable liquid, e.g. with saline or 5% glucose solution, to give a ready-for-use-formulation, which e.g. can be administered as infusion or injection. Alternatively (but less preferred) the concentrate may be used to be administered directly. Generally, in the art concentrates are also referred to as “Parenteralia diluenda”.

An alternative parenteral formulation suitable for use in the present disclosure can be a “ready-to use” formulation. The term “ready-to-use” in the context of the present disclosure typically means that no further preparation step is necessary before administering the parenteral formulation to the patient, for example by injecting the formulation. Moreover, there is no need to add further additives or solvents, such as water, for injection before administration of the parenteral formulation.

The parenteral formulation of the present disclosure preferably contains siponimod in a concentration of 0.05 to 3.5 mg/mL, preferably 0.1 to 2.0 mg/mL, more preferably 0.015 to 1.5 mg/mL. In a particularly preferred embodiment the parenteral formulation being present in form of a concentrate can contain siponimod in concentrations of 0.25 mg/mL, 0.5 mg/mL or 1.0 mg/mL, especially 1 mg/mL.

As far as the before-described concentration of siponimod is concerned, this applies to a parenteral formulation being present as a concentrate; i.e. in not further diluted form. It is evident that the concentration gets smaller, if the concentrate is further diluted for example to form an infusion solution.

The parenteral formulation used in the present disclosure, preferably being in the form of a concentrate, comprises

(A) Siponimod in a concentration of 0.05 to 3.5 mg/mL, preferably of 0.1 to 2.0 mg/mL, more preferably 0.015 to 1.5 mg/mL, in particular 1.0 mg/mL;

(B) hydroxypropyl-8-cyclodextrin in a concentration of 50 to 300 mg/mL, preferably of 65 to 200 mg/mL, more preferably 80 to 150 mg/mL, in particular about 100 mg/mL;

(C) mannitol in a concentration of 5 to 200 mg/mL, preferably of 10 to 100 mg/mL, more preferably 20 to 80 mg/mL, in particular 30 mg/mL;

(D) 2-amino-2-(hydroxymethyl)propan-1,3-diol in a concentration of 0.2 to 2.0 mg/mL, preferably of 0.3 to 1.5 mg/mL, more preferably 0.4 to 1.0 mg/mL, even more preferably 0.5 to 0.8 mg/mL, in particular about 0.60 mg/mL, i.e. 5 mM; and

(E) water.

Formulations Storage Conditions: The siponimod film-coated tablets, as well as other available tablet and capsule formulations and oral solutions prepared at the site pharmacy, should be stored refrigerated at 2 to 8° C. The concentrate for solution for infusion is to be stored refrigerated at 2 to 8° C.

### Clinical Study

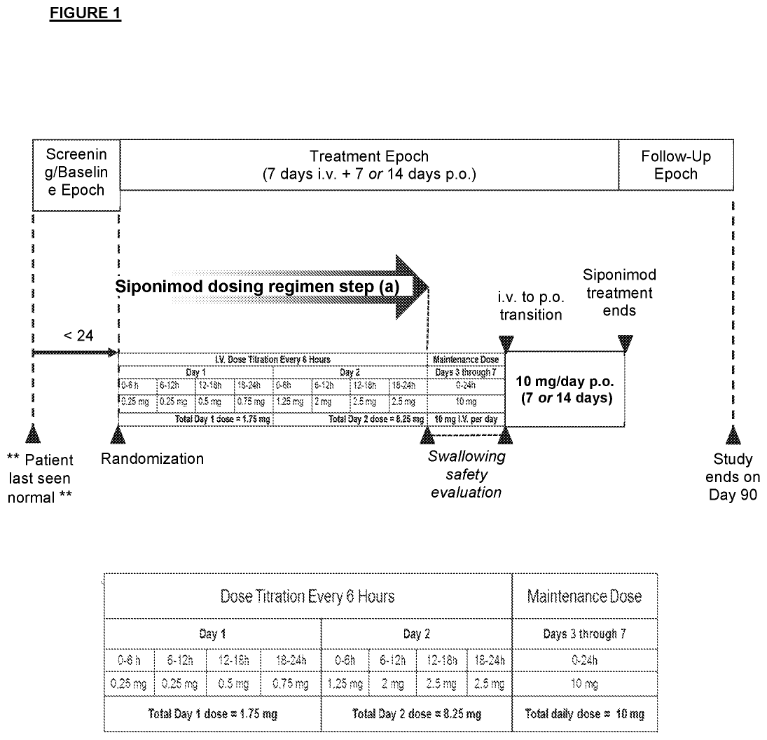

The clinical study investigates the initial efficacy and safety of siponimod administered on top of standard-of-care compared to placebo in patients with intracerebral hemorrhage (ICH). This is a randomized, doubled-blinded, placebo-controlled, parallel group study of siponimod on top of standard-of-care for ICH, consisting of 3 epochs: Screening/Baseline, Treatment, and Follow-Up (see FIG. 1).

### 1. Objectives and Endpoints

### 1.1 Primary Objective(s)

| | Endpoints related to |
|---|---|
| Primary objective(s) | primary objective(s) |
| To obtain the first efficacy estimate | Modified Rankin Scale |
| of 10 mg siponimod daily (7 days i.v. | (mRS) on Day 90. |
| with titration followed by 7 days p.o.) | |
| compared to placebo on improving | |
| global functioning measured by the | |
| modified Rankin Scale (mRS) on | |
| Day 90 after ICH. | |

### 1.2 Secondary Obiective(s)

| Secondary | Endpoints related to |
|---|---|
| objective(s) | secondary objective(s) |
| To assess the safety profile | Continuous assessment of AEs/SAEs |
| of siponimod in ICH patients. | during the course of the study (90 days). |

### 1.3 Exploratory Objective(s)

| | Endpoints related to |
|---|---|
| Exploratory objective(s) | exploratory objective(s) |
| To compare early neurological deterioration | NIHSS on Days 1-7. |
| (END), defined as National Institutes of | |
| Health Stoke Scale (NIHSS, or derived | |
| mNIHSS) worsening by ≥ 4 points, between | |
| placebo and Siponimod, on Day 7 after ICH. | |
| To evaluate the efficacy of siponimod on the | CT scans at admission (standard-of-care), |
| development of perihematomal edema | 24-48 hours after the initial scan |
| (PHE), both absolute (aPHE) and relative | (standard-of-care), Day 7 (study), and |
| (rPHE) volumes, measured by CT | Day 14 (study). |
| neuroimaging between 24 and 48 hours after | |
| ICH (standard of care), and on Days 7 and 14 | |
| (study assessments) after ICH. | |
| To measure the trajectory of recovery over | Modified Rankin Scale (mRS) at |
| time between Day 14 and Day 90 using | Days 14, 30, and 90. |
| changes in mRS scores. | |
| To evaluate the efficacy of siponimod in ICH | NIHSS at Day 90. |
| measured by the NIHSS on Day 90. | |
| To determine if CYP2C9 genotype influences | CYP2C9 genotype. |
| siponimod pharmacokinetics. | |

### 2. Screeninq/Baseline Epoch

The screening/baseline epoch lasts no longer than 24 hours from the time of onset of ICH, defined as the time the patient was last witnessed to be at their normal neurological baseline, and consists of:
The initial diagnostic neuroimaging study (CT or MRI) to determine the cause of stroke
Determining the Glasgow Coma Scale (GCS, http://www.glasgowcomascale.org/) score on presentation
Obtaining medical history, including current medications
Hospital admission laboratory studies
Electrocardiogram (ECG)
Pregnancy test for premenopausal female patients
Vital signs and physical examination, including neurological examination, and
Determination of NIH Stroke Scale (NIHSS, https://www.ninds.nih.gov/Stroke-Scales-and-Related-Information) score on presentation.

### 3. Treatment Epoch

Patients fulfilling all eligibility criteria are randomly allocated to one of two treatment groups in a ratio of 1:1. The treatment starts as soon as possible but no later than 24 h after the time of onset of the ICH, defined as the time the patient was last witnessed to be healthy, defined as functioning at their normal, pre-event neurological baseline.
The total treatment lasts 14 days (see FIG. 1):
   7 days of i.v. siponimod with titration to the final daily dose of 10 mg/day; during the 7 days of i.v. infusion treatment, all patients undergo a swallowing safety evaluation per the treating hospital's institutional guidelines and practices.
   If the patients pass a swallowing safety evaluation, 7 days of 10 mg siponimod p.o. QD.
   Patients who do not successfully pass a swallowing safety evaluation are not transitioned to the p.o. phase of treatment, and siponimod is discontinued after Day 7; but they are not terminated from the study. These patients continue to be followed for the remainder of the assessment schedule (Table 3).
      i.v. Dose Titration

The dose titration schedule is based on estimations of the cardiovascular effects of siponimod balanced with the therapeutic need to achieve fast, effective siponimod concentrations in ICH patients, where the timely achievement of expected therapeutic concentrations is of great importance.

The siponimod i.v. dosing regimen is as follows:
Day 1: 0.25 mg over 6 hours (×2), then 0.5 mg over 6 hours, then 0.75 mg over 6 hours for a total Day 1 dose of 1.75 mg
Day 2: 1.25 mg over 6 hours, then 2 mg over 6 hours, then 2.5 mg over 6 hours (×2) for a total Day 2 dose of 8.25 mg
Days 3 through Day 7: 2.5 mg over 6 hours (×4) for a total daily dose of 10 mg.
If patient can swallow then the administration from Day 8 to Day 14 is oral.

During the i.v. up-titration period patients are closely monitored. Continuous cardiac monitoring is implemented in the Stroke Unit/Intensive Care Unit setting (telemetry or bedside monitoring) in all patients during days indicated in the assessment schedule (Table 4; see below). Monitoring starts from 1 hour before the first dose of siponimod and continues up to at least 48 hours after the first dose administration. Continuous cardiac monitoring is done for a longer duration on a case-by-case basis at the discretion of the Investigator and/or treating intensivist. Cardiac safety monitoring data are used for cardiac rhythm evaluation (mainly bradyarrhythmias, such as atrioventricular blocks and sinus pauses) and for HR assessment (bradycardia). Bradycardia and/or bradyarrhythmias with siponimod administration typically occur within the first 48 hours of dosing, and are almost completely eliminated with siponimod up-titration as claimed by the present disclosure. In case of bradycardia is markedly symptomatic, or inappropriate for the clinical condition in the judgement of the treating intensivist or in case of cardiac rhythm abnormalities (e.g. AVB or SP) the i.v. administration of siponimod, the i.v. infusion, is interrupted.

Bradycardia with S1P modulators is usually benign, transient, and does not require treatment (Schmouder et al. 2012). The patient is assessed to determine if treatment continuation is acceptable to the treating physician and the Investigator (e.g., 1st or 2nd degree AV blocks) and treatment is continued once the patient recovers from symptomatic bradycardia. In the case of 3rd degree AV block and/or a hemodynamically-affected patient, the treatment is not reinitiated.

Any reduction in heart rate, which, in the opinion of the Investigator or treating intensivist, is clinically significant and requires intervention (e.g., acutely altered mental status, ongoing severe ischemic chest pain, congestive heart failure, hypotension, or other signs of shock) is treated according to standard medical practice, and suggested treatment would include: (i) Anticholinergics (e.g. atropine subcutaneous or i.v.) or (ii) Beta-agonists/sympathomimetics (e.g. dopamine or epinephrine). Dosing of these is individualized with respect to the desired clinical effect by the treating intensivist.

p.o. Dose

Eligible patients who pass a swallowing safety evaluation continue with 7-day p.o. phase of treatment with siponimod 10 mg QD. During the Treatment Epoch, all patients undergo study-specific assessments according to the Assessment Schedule (Table 4 in the example section).

### 4. Efficacy/Pharmacodynamics

### 4.1 Clinical Outcome Assessments (COAs)

4.1.1 Modified Rankin Scale (mRS)

The modified Rankin Scale (mRS), is a widely-used, clinician-assessed instrument, and is considered the current standard assessment for stroke outcomes by most Health Authorities. It consists of 6 grades of disability, higher scores indicating more severe disability (0=asymptomatic, 6=dead).

| | |
|---|---|
| 0 | No symptoms |
| 1 | No significant disability. Able to carry out all usual activities, despite |
| 2 | Slight disability. Able to look after own affairs without assistance, |
| | but unable to carry out all previous activities |
| 3 | Moderate disability. Requires some help, but able to walk unassisted |
| 4 | Moderately severe disability. Unable to attend to own bodily needs |
| | without assistance, and unable to walk unassisted |
| 5 | Severe disability. Requires constant nursing care and attention, |
| | bedridden, incontinent |
| 6 | Dead |

(Stroke. 2017; 48-2017 American Heart Association, Inc.-Joseph P. Broderick, et al.)

The strength of the mRS is that it captures the full spectrum of limitations in activity and participation after stroke. The inter-rater reliability of the scale is moderate and improves significantly with structured interviews (0.56 versus 0.78; Banks and Marotta 2007); and this structured approach is used in our study (Wilson et al. 2002, Wilson et al. 2005). The mRS is administered by investigators, study nurses or research assistants. Training in administration of the structured mRS interview is provided to site personnel as necessary, and proficiency certification is monitored and centrally recorded. In this study, structured mRS interviews is video recorded, then securely transferred to and rated by a Central Independent Adjudication Panel. Individual (rater) mRS scores (and the panel average) as well as the panel consensus score for each interview is recorded.

The mRS score at 90 days after ICH is the primary endpoint for measuring siponimod efficacy in this study.

The 90-day mRS score has been used as an endpoint in many stroke studies, including the INTERACT2 (Anderson et al. 2013), ATACH (Qureshi et al. 2010), SAMURAI-ICH (Koga et al. 2014), and ENOS (ENOS Trial Investigators 2015) trials. In addition, a recent study (Murthy et al. 2015) using the mRS found an association between early PHE expansion after ICH and poorer 90 day functional outcomes in basal ganglia hemorrhages <30 cc, which is the population chosen for the clinical trial of the present disclosure.

4.1.2 NIH Stroke Scale (NIHSS)

The National Institutes of Health Stroke Scale (NIHSS), is the most widely used clinical instrument to assess the neurological impact of acute stroke (Lyden 2017). The NIHSS consists of 13 individually scored items, with a maximal composite score of 42, higher scores indicating greater stroke severity. The NIHSS is administered by investigators or study nurses. NIHSS training certification is monitored and centrally recorded.

Patients with ICH often experience early neurological deterioration (END) within the first few days after stroke due either to expansion of the initial hematoma or increase in PHE.

Both increased total peripheral white blood cell counts (Sun et al. 2012) and greater absolute PHE volume (Rodriguez-Luna et al. 2016) have been shown to be associated with END. Various studies and centers define END criteria using different rating scales (GCS, NIHSS) and as occurring within different time windows (24 hours to 7 days) after ICH. For the study of the present disclosure, END is defined as NIHSS worsening by 4 or more points between initial presentation and Day 7 after ICH.

### 5. Safety

5.1 Electrocardiogram (ECG)

Continuous cardiac monitoring is implemented via bedside monitoring in all patients during days when the patient is in the stroke/intensive care unit. Cardiac monitoring is performed from 1 hour before dosing and up to 48 hours after the first drug administration. Continuous cardiac monitoring is done for a longer duration on a case-by-case basis, depending on the patient's conditions. Standard twelve-lead ECGs is performed for all patients at the time points as indicated in Table 3.

Cardiac safety monitoring data is used for cardiac rhythm evaluation (mainly bradyarrhythmias, such as atrioventricular blocks and sinus pauses: Frequency and duration of sinus pauses (>2 seconds)) and for heart rate (HR) assessments.

6. Other assessments

6.1 CYP2C9 Genotyping

Genotyping is performed to determine whether CYP2C9 genotype influences siponimod pharmacokinetics.

6.2 CT Scan

Studies of the trajectory of perihematomal edema (PHE) assessed with either magnetic resonance (MR) (Venkatasubramanian et al. 2011) or computed tomography (CT) imaging (Staykov et al. 2011) largely agree on the average time course of its development, which increases to a plateau between days 7-14 after ICH. Analyzing data from the VISTA-ICH archive (Murthy et al., 2015) showed an association between early PHE expansion within 72 hours after ICH and poorer 90 day functional outcomes in basal ganglia hemorrhages 30 cc in volume.

In this study, following the initial diagnostic CT, repeated CT images is obtained between 24-48 hours after the diagnostic scan (i.e., standard-of-care in ICH), and on Days 7 and 14 after ICH to capture the trajectory of PHE increase and plateau after ICH. Only noncontrast CT scans are obtained on Days 7 and 14. In the analysis it is used the noncontrast scan acquired on each patient at first follow-up (24-48 hours after the diagnostic scan). However it does not dictate the standard-of-care scan protocol for the initial and first follow-up scan beyond ensuring a noncontrast scan is acquired, as it usually happen according to standard practice.

6.3 Actigraphy

The use of wearable or externally-monitored actigraphy in a variety of neurological and musculoskeletal disorders, including stroke rehabilitation, is growing; and wearable devices, which may or may not provide direct patient feedback, are increasingly used to measure functional mobility and rehabilitation outcomes (Wang et al. 2017). The actigraphy devices are similar to a wrist-watch and are lightweight, water-resistant, and can be worn continuously for several days. To measure functional mobility with greater sensitivity, and in a more naturalistic (e.g., home) setting, patients of the study of the present disclosure are fitted with wrist-worn actigraphy devices around Days 14, 30, and 90 after ICH.

### GENERAL TERMS

Throughout the description and claims of this specification, the words “comprise” and “contain” and variations of them mean “including but not limited to” and they are not intended to (and do not) exclude other moieties, additives, components, integers or steps.

Throughout the description and claims of this specification, the singular encompasses the plural unless the context otherwise requires. In particular, where the indefinite article is used, the specification (which term encompasses both the description and the claims) is to be understood as contemplating plurality as well as singularity, unless the context requires otherwise.

Features, integers, characteristics, compounds, chemical moieties or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings) and/or all of the steps of any method or process so disclosed may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

The term “treatment” includes: (1) preventing or delaying the appearance of clinical symptoms of the state, disorder or condition developing in an animal, particularly a mammal and especially a human that may be afflicted with or predisposed to the state, disorder or condition but does not yet experience or display clinical or subclinical symptoms of the state, disorder or condition; (2) inhibiting the state, disorder or condition (e.g. arresting, reducing or delaying the development of the disease or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment. In the specific context of stroke treatment, most preferably the treatment starts as soon as possible after the time of onset of ICH symptoms.

The “time of onset of ICH” is defined as the time the patient was last witnessed healthy or at their pre-event neurological baseline if their prior neurological status was not normal. “Treat,” “treatment,” “therapeutic treatment” or “treating,” as used herein, refers to administering an active agent for therapeutic purposes, in particular, it means, for example, obtaining beneficial or desired results, such as clinical results, in the reduction of inflammation, edema formation and other post-stroke secondary injuries.

One aspect of the treatment is, for example, that the treatment should have a minimal adverse effect on the patient, e.g. the agent used should have a high level of safety, for example, without producing the side effects of the known S1P receptor modulator treatment regimens, such the negative chronotropic effect, liver enzyme elevation or excessive lymphophenia.

The expression “introducing a siponimod treatment” as used herein means administering an initial titration regimen of siponimod, followed by administering a respective maintenance regimen.

As used herein the term “dose” has its general meaning in the art, wherein preferred embodiments are as defined herein. The term dose refers to a specified amount of medication taken at one time (e.g. 0.25 mg of siponimod administered as a first dose), wherein the amount of medication is calculated on the basis of the weight of active ingredient in its free form. It is the amount or quantity of medicine to be taken or administered to the patient every time (e.g. every 6 hours) in a day.

As used herein, the term “first dose” has its general meaning in the art, wherein preferred embodiments are as defined herein. The “first dose” of siponimod is the first administered dose on Day 1 of the treatment.

As used herein the term “maintenance daily dose” has its general meaning in the art, wherein preferred embodiments are as defined herein. The “maintenance daily dose” of siponimod is the dose administered in step (b) of the method of treatment of the present disclosure. As used herein the term “dosing regimen” refers to the treatment plan specifically indicating the administering pattern of a drug over a period of time. The dosing regimen defines the amount of a drug and the number and frequency of its administrations over a specified period of time that is employed in the treatment of a disease. A close adherence to the dosing regimen is important for achieving a therapeutic effect of the drug and maintaining the therapy safe. The potential consequences of noncompliance are loss of the therapeutic effect and/or an increased risk of adverse events. The dosing regimen would be explained for example in the “dosage and administration” section or “posology and method of administration” section of labeling for human prescription drugs.

As used herein the term “dosage form” has its general meaning in the art, wherein preferred embodiments are as defined herein. The term “dosage form” describes the physical characteristics of a drug product—e.g., tablet, capsule or solution—which contains the drug substance and almost invariably other ingredients, such as excipient, fillers, flavours, preservatives, emulsifiers, etc. The term dosage form indicates the unit doses. Dosage forms are pharmaceutical drug products in the form in which they are marketed for use, with a specific mixture of active ingredients and inactive components (excipients), in a particular configuration (such as for example a capsule, tablets, ointments, liquid solutions, powder, etc.), and apportioned into a particular dose.

As used herein the term “AV blocks” or the abbreviation “AVB” as used herein means “atrioventricular block”.

The abbreviation “SP” as used herein means “sinus pause”, also known as sinoatrial arrest is a medical condition wherein the sinoatrial node of the heart transiently ceases to generate the electrical impulses that normally stimulate the myocardial tissues to contract and thus the heart to beat. It is defined as lasting from 2.0 seconds to several minutes.

The abbreviation “QT” is a measure of the time between the start of the Q wave and the end of the T wave in the heart's electrical cycle. QTcf is the an alternative correction formula using the cube-root of RR, i.e. QTcF=(QT)/(cube-root of RR).

The abbreviation “PR rate” as used herein has its general meaning in the art, wherein preferred embodiments are as defined herein. In electrocardiography, the PR interval is the period, measured in milliseconds, that extends from the beginning of the P wave (the onset of atrial depolarization) until the beginning of the QRS complex (the onset of ventricular depolarization); it is normally between 120 and 200ms in duration. The PR interval is sometimes termed the PQ interval.

The term “resting heart rate” (RHR) as used herein means the number of contractions of the heart that occur in a single minute while the body is at complete rest. This number will vary depending upon the age, gender, and general health of a person.

The term “baseline heart rate” as used herein means a referential heart rate to which other heart rates, such as the heart rate under chronic beta-blocker treatment, can be compared to. Typically, the RHR in the absence of any heart rate-affecting medication serves as the baseline heart rate. As used herein, “bradycardia” typically refers to a RHR <50 bpm. The abbreviation “HR” as used herein means “heart rate”. A person having ordinary skill in the art will typically measure the HR using an electrocardiograph. The expression “Eₘₐₓ,” as used herein means the maximum change from baseline in time matched, hourly average HR.

As used herein the term “absolute perihematomal edema volume (aPHE)” and “relative perihematomal edema volume” (rPHE) have their general meaning in the art, wherein preferred embodiments are as defined herein. Relative perihematomal (rPHE) volume is defined as absolute edema perihematomal (aPHE) volume divided by hematoma volume, yielding a unitless ratio variable. Absolute edema volume is measured by computer-assisted volumetric measurement technique known in the art.

As used herein the term “ABC/2” has its general meaning in the art. ABC/2 is a fast and simple method for estimating the volume of intracerebral haemorrhage (or any other ellipsoid lesion for that matter) which does not require volumetric 3D analysis or software. Intracerebral haemorrhage volume is an important predictor of morbidity and mortality (and thus trial eligibility) which is often under-reported. It has been well-validated and correlates highly with volumes calculated by planimetric techniques. The formula for the calculation of ABC/2 was first described by Kwak et al. (Kwak R, Kadoya S, Suzuki T. Factors affecting the prognosis in thalamic hemorrhage. Stroke. 14 (4): 493-500) and popularised by Kothari et al. (Kothari R U, Brott T, Broderick J P, Barsan W G, Sauerbeck L R, Zuccarello M, Khoury J. The ABCs of measuring intracerebral hemorrhage volumes. Stroke. 27 (8): 1304-5).

The formula is: A×B×C/2, wherein
A=greatest haemorrhage diameter in the axial plane
B=haemorrhage diameter at 90° to A in the axial plane
C=originally described as the number of CT slices with haemorrhage multiplied by the slice thickness, but can simply be substituted with the craniocaudal diameter of the haemorrhage where there is access to multiplanar reformats.

If the measurements are made in centimetres (cm), then the volume will be in cubic centimetres (cm³). The above formula is a simplified version of the formula for the volume of an ellipsoid, which is: 4/3Π×(A/2)×(B/2)×(C/2), where A, B and C are the three diameters of the ellipsoid. If Π is estimated as 3, then the formula can be simplified to ABC/2.

As used herein the term “CYP2C9 poor metabolizer” or “ poor metabolizer” , such as CYP2C9*2*3 and CYP2C9*2*3 “poor metabolizer” or “poor metabolizer genotype” includes patients who experience a significantly higher exposure following siponimod administration than normal patients at a given drug dose e.g. 2 mg once daily of siponimod. The poor metabolizer genotype may include the subtype(s) of the CYP2C9 genotype associated with poor metabolism of 1-{4-[(1E)-N-{[4-cyclohexyl-3-(trifluoromethyl)benzyl]oxy}ethanimidoyl]-2-ethylbenzyl}-3-azetidine carboxylic acid. The poor metabolizer genotype includes the CYP2C9*3*3 and CYP2C9*2*3 genotypes, for example the CYP2C9*3*3 genotype.

The term “pharmaceutical composition” is defined herein to refer to a mixture or solution containing at least one active agent (also referred to as “active ingredient” or therapeutic agent) to be administered in order to treat a particular disease or condition, in particular to treat stroke, in particular ICH. In another embodiment, the term “pharmaceutical composition” is defined herein to refer to a mixture or solution containing at least one active agent (i.e. “active ingredient” or therapeutic agent) to be administered in order to prevent a particular disease or condition, in particular to prevent or delay the onset or development or progression of a stroke such as ICH. The pharmaceutical composition can be formulated for particular routes of administration such as oral or topical administration.

As used herein the term “co-crystal” indicates a crystalline material composed of two or more different molecules within the same crystal lattice that are associated by nonionic and noncovalent bonds and that generally are in a stoichiometric ratio. In the pharmaceutical field a co-crystal is generally defined as a crystalline materials composed of two or more different molecules, typically drug and co-crystal formers (“coformers”), in the same crystal lattice. Co-crystals are readily distinguished from salts because unlike salts, their components are in a neutral state and interact non-ionically. In addition, co-crystals differ from polymorphs, which are defined as including only single-component crystalline forms that have different arrangements or conformations of the molecules in the crystal lattice, amorphous forms, and multicomponent phases such as solvate and hydrate forms. Instead co-crystals are more similar to solvates, in that both contain more than one component in the lattice. From a physical chemistry perspective, co-crystals can be viewed as a special case of solvates and hydrates, wherein the second component, the coformer, is nonvolatile. Therefore, co-crystals are classified as a special case of solvates in which the second component is nonvolatile. Co-crystals can be tailored to enhance drug product bioavailability and stability and to enhance the processability of active pharmaceutical ingredients (APIs) during drug product manufacture. In a preferred embodiment siponimod is added to the formulation in form of a co-crystal.

As used herein the term “salts” has its general meaning in the art, wherein preferred embodiments are as defined herein. Examples of pharmaceutically acceptable salts of siponimod include salts with inorganic acids, such as hydrochloride, hydrobromide and sulfate, salts with organic acids, such as acetate, fumarate, hemifumarate, maleate, benzoate, citrate, malate, methanesulfonate and benzenesulfonate salts, or, when appropriate, salts with metals such as sodium, potassium, calcium and aluminium, salts with amines, such as triethylamine and salts with dibasic amino acids, such as lysine. In a preferred embodiment siponimod is in the hemifumarate salt form. The compounds and salts of the combination of the present invention encompass hydrate and solvate forms. In a preferred embodiment siponimod is added to the formulation in form of an acid addition product with fumaric acid.

As used herein, the term “combination”, “pharmaceutical combination”, “fixed combination”, “non-fixed combination”, “co-administration”, “combined administration” or the like has its general meaning in the art, wherein preferred embodiments are as defined herein. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the invention and a co-agent, are both administered to a patient simultaneously in the form of a single entity or dosage. The term “non-fixed combination” means that the active ingredients, e.g. a compound of the invention and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### EXAMPLES

The following Examples serve to illustrate the disclosure without limiting the scope thereof, while they on the other hand represent preferred embodiments of the reaction steps, inter-mediates and/or the process of the present disclosure.

1. Preparation of Siponimod Parenteral Formulations

### Example 1

884.2 g trehalose were added to 18000 mL milliQ water and the mixture was stirred until complete dissolution. 12.0 g 2-amino-2-(hydroxymethyl)propan-1,3-diol (Tris, Trometamol) were added and the mixture was stirred until complete dissolution. 100 g polyoxyethylen(20)-sorbitan-monooleate (Tween 80, Polysorbat 80) were added and the mixture was stirred until complete dissolution. 5.56 g (accurately weighted) of siponimod hemifumarate were added and the mixture was stirred until complete dissolution. The pH of the solution was adjusted to a value of 8.0 ±0.1. MilliQ water was added until a total weight of 20.28 g and the mixture was stirred to obtain a homogenous solution. The solution was filtered through a 0.22 μm PVDF filter and the first 5000 mL of the filtrate were discarded. The solution was filled into 6R clear vials.

| Composition | Quantity in mg/mL |
|---|---|
| 1-{4-[(1E)-N-{[4-cyclohexyl-3- | 0.278 |
| (trifluoromethyl)benzyl]oxy}ethanimidoyl]- | |
| 2-ethylbenzyl}-3-azetidinecarboxylic | |
| acid/fumaric acid (2:1) co-crystal | |
| Trehalosedihydrate | 44.21 |
| Polyoxyethylen(20)-sorbitan-monooleat | 5.0 |
| 2-amino-2-(hydroxymethyl)propan-1,3-diol | 0.6 |
| 1N HCl or 1N NaOH | q.s to pH 8.0 |
| Water | q.s |

The product was lyophilized according to the following cycles:
Lyophilization cycle parameters: lyophilization program for siponimod formulation

| | | | Shelf | Chamber |
|---|---|---|---|---|
| Step | Operation | Time [hh:mm] | Temperature | Pressure |
| 1 | Vial loading | As required | 15° | Ambient |
| 2 | Hold | 0:05 | 15° C. | Ambient |
| 3 | Freeze ramp | 00:55 | −40° C. | Ambient |
| | | (1.0° C./min) | | |
| 4 | Freeze hold | 2:00 | −40° C. | Ambient |
| 5 | Freeze hold | 1:00 | −40° C. | 0.2 mBar |
| 6 | Freeze ramp | 04:00 | −20° C. | 0.2 mBar |
| | | (0.083° C./min) | | |
| 7 | Freeze hold | 80:00 | −20° C. | 0.2 mBar |
| 8 | Freeze ramp | 13:30 | −0° C. | 0.1 mBar |
| | | (0.025° C./min) | | |
| 9 | Freeze hold | 10:00 | −0° C. | 0.1 mBar |
| 10 | Secondary | 16:30 | 25° C. | 0.1 mBar |
| | drying | (0.025° C./min) | | |
| | Rate | | | |
| 11 | Secondary | 10:00 | 25° C. | 0.1 mBar |
| | drying | | | |
| | hold | | | |
| 12 | Secondary | 1:00 | 25° C. | 0.1 mBar |
| | drying | | | |
| | hold | | | |
| 13 | Storage | As required/until | 15° C. | 0.1 mBar |
| | | vacuum release and | | |
| | | stoppering | | |
| 14 | Stoppering | | 20° C. | 850 ± 50 |
| | | | | mbar |

The apparatus used for lyophilisation was “VIRTIS GENESIS 25EL” from SP scientific.

For reconstitution water for injection was used.

### Example 2

250 mL milliQ water were transferred into a suitable glass bottle and 50 g hydroxypropyl β-cyclodextrin were added. The mixture was stirred for 30 minutes at 500 rpm and a clear solution was formed. 556 mg (accurately weighted) of 1-{4-[(1E)-N-{[4-cyclohexyl-3-(trifluoromethyl)benzyl]oxy}ethanimidoyl]-2-ethylbenzyl}-3-azetidinecarboxylic acid/fumaric acid (2:1) co-crystal were added and the mixture was stirred for 15 minutes at 500 rpm and a suspension was formed. 305 mg 2-amino-2-(hydroxymethyl)propan-1,3-diol (Tris, Trometamol) were added and the mixture was stirred for 60 minutes at 500 rpm and a clear solution having a pH value of 7.897 was formed. 250 μl of 1N NaOH were added and after stirring for 2 minutes at 500 rpm a clear solution having a pH value of 7.983 was formed. 15 g mannitol were added and the mixture was stirred for 15 minutes at 500 rpm and a clear solution was formed. MilliQ water was added to fill up to a volume of 500 mL of a clear solution having a pH value of 8.015. The solution filtered through a 0.22 μm PVDF filter and the first 20 mL of the filtrate were discarded. The solution was filled into 6R clear vials. The 6 mL amber glass vial and the grey rubber stopper, aluminium flip-off cap nature/nature has been autoclaved at 121° C. for 30 minutes prior to filling. The vials were stored at 2 to 8° C. until use, each vial containing:

| Siponimod Composition for | Quantity |
|---|---|
| i.v. administration | in mg/mL |
| 1-{4-[(1E)-N-{[4-cyclohexyl-3- | 1.112 |
| (trifluoromethyl)benzyl]oxy}ethanimidoyl]- | |
| 2-ethylbenzyl}-3-azetidinecarboxylic acid/ | |
| fumaric acid (2:1) co-crystal | |
| hydroxypropyl β-cyclodextrin | 100 |
| Mannitol | 30 |
| 2-amino-2-(hydroxymethyl)propan-1,3-diol | 0.61 |
| 1N HCl or 1N NaOH | q.s to pH 8.0 |
| Water | q.s |

### Example 3

250 mL milliQ water were transferred into a suitable glass bottle and 50 g hydroxypropyl β-cyclodextrin were added. The mixture was stirred for 30 minutes at 500 rpm and a clear solution was formed. 556 mg (accurately weighted) of 1-{4-[(1E)-N-{[4-cyclohexyl-3-(trifluoromethyl)benzyl]oxy}ethanimidoyl]-2-ethylbenzyl}-3-azetidinecarboxylic acid/fumaric acid (2:1) co-crystal were added and the mixture was stirred for 15 minutes at 500 rpm and a suspension was formed. 305 mg 2-amino-2-(hydroxymethyl)propan-1,3-diol (Tris, Trometamol) were added and the mixture was stirred for 60 minutes at 500 rpm and a clear solution having a pH value of 7.878 was formed. 250 μl of 1N NaOH were added and after stirring for 2 minutes at 500 rpm a clear solution having a pH value of 7.997 was formed. 3 g sodium chloride were added and the mixture was stirred for 15 minutes at 500 rpm and a clear solution a pH value of 8.112 was formed. 220 μl of 1N HCl were added and after stirring for 10 minutes at 500 rpm a clear solution having a pH value of 8.004 was formed. MilliQ water was added until a volume of 500 mL of a clear solution having a pH value of 8.002 was formed. The solution filtered through a 0.22 μm PVDF filter and the first 20 mL of the filtrate were discarded. The solution was filled into 6R clear vials. The 6 mL amber glass vial and the grey rubber stopper, aluminium flip-off cap nature/nature has been autoclaved at 121° C. for 30 minutes prior to filling. The vials were stored at 2 to 8° C. until use, each vial containing:

| Siponimod Composition for | Quantity in |
|---|---|
| i.v. administration | mg/mL |
| 1-{4-[(1E)-N-{[4-cyclohexyl-3- | 1.112 |
| (trifluoromethyl)benzyl]oxy}ethanimidoyl]- | |
| 2-ethylbenzyl}-3-azetidinecarboxylic acid/ | |
| fumaric acid (2:1) co-crystal | |
| hydroxypropyl β-cyclodextrin | 100 |
| Sodium chloride | 6 |
| 2-amino-2-(hydromethyl)propan-1,3-diol | 0.61 |
| 1N HCl or 1N NaOH | q.s to pH 8.0 |
| Water | q.s |

### 2. Clinical Study

A previously conducted absolute bioavailability study up to 1 mg/24 hours in healthy volunteers used the i.v. route of administration (CBAF312A2126). The safety of the i.v. route in humans was supported by a local tolerance study in rabbit and cardiovascular safety studies in guinea pig, rat and rabbit using i.v. (bolus) route. Cₘₐₓ-related transient cardiovascular effects (in line with the expected pharmacology) were identified in the i.v. cardiovascular safety studies and were similar to the effects identified by oral route.

1. Study Objectives

1.1. Primary objective

The primary objective is to demonstrate the efficacy of a two week treatment with siponimod administered daily (7 days i.v. with titration followed by 7 days p.o.) compared to placebo on improving global functioning measured by the modified Rankin Scale (mRS) score on Day 90 after ICH.

1.2. Secondary objectives

The first key secondary objective is to demonstrate the safety of siponimod in patients suffering from ICH. The endpoint related to this secondary objective is a continuous assessment of adverse effects/severe adverse effects (AEs/SAEs) during the course of the study (90 days).

1.3. Exploratory objectives include

To compare early neurological deterioration (END), defined as National Institutes of Health Stoke Scale (NIHSS, or derived mNIHSS) worsening by 4 points, between placebo and siponimod, on Day 7 after ICH. The endpoint related to this exploratory objective is the measurement of NIHSS on days 1 to 7.

To demonstrate the efficacy of siponimod on the development of perihematomal edema (PHE), both absolute (aPHE) and relative (rPHE) volumes, measured by CT neuroimaging between 24 and 48 hours after ICH (standard of care), and on Days 7 and 14 (study assessments) after ICH. The endpoint related to this exploratory objective is the CT scans at admission (standard-of-care), 24-48 hours after the initial scan (standard-of-care), Day 7 (study), and Day 14 (study).

To measure the trajectory of recovery over time between Day 14 and Day 90 using changes in mRS scores. The endpoint related to this exploratory objective is the modified Rankin Scale (mRS) at Days 14, 30, and 90.

To demonstrate the efficacy of siponimod in ICH measured by the NIHSS on Day 90. The endpoint related to this exploratory objective is the measurement of the NIHSS at Day 90.

### 2. Population

The study population consists of adult patients with stroke due to ICH fulfilling the eligibility criteria listed below. Approximately 50 patients per treatment group (100 patients total) are randomized, with an expected drop-out rate of approximately 20% to have approximately 80 completers (Day 90).

2.1. Inclusion Criteria Exclusion Criteria

ICH patients eligible for inclusion in this study fulfill all of the following criteria:
2.1.1. Male or female patients aged 18 to 80 years (inclusive).
2.1.2. Written informed consent obtained before any study assessment is performed.
2.1.3. If the patient is not able to give the informed consent personally, consent by a relative or legal representative is acceptable.
2.1.4. Spontaneous, supratentorial intracerebral hemorrhage in deep brain structures (putamen, thalamus, caudate, and associated deep white matter tracts) with a volume ≥10 mL but ≤30 mL (calculated by the ABC/2 method, after Kothari et al 1996) determined by routine clinical MRI or CT.

2.1.5. Patients with the onset of ICH witnessed and/or last seen healthy no longer than 24 hours previously.
2.1.6. Patients with Glasgow Coma Scale (GCS) best motor score no less than 6.

2.2. Exclusion Criteria

ICH patients fulfilling any of the following criteria are not eligible for inclusion in this study: Use of other investigational drugs within 5 half-lives of enrollment, or until the expected pharmacodynamic effect has returned to baseline (for biologics), whichever is longer. 1

2.2.1. History of hypersensitivity to any of the study drugs or to drugs of similar chemical classes (e.g., fingolimod).
2.2.2. Current use of concomitant medications with potent CYP2C9/3A4 inhibitory or induction potential.
2.2.3. Necessity for mechanical ventilation at screening.
2.2.4. Infratentorial (midbrain, pons, medulla, or cerebellum) or superficial cortical (lobar) ICH.
2.2.5. Candidates for surgical hematoma evacuation or other urgent surgical intervention (i.e., surgical relief of increased intracranial pressure) on initial presentation. If during the treatment period surgical hematoma evacuation or surgical intervention to lower intracranial pressure becomes indicated, the investigational treatment should be stopped.
2.2.6. Patients with intraventricular hematoma extension, with or without hydrocephalus, on initial presentation.
2.2.7. Secondary ICH due to:
   aneurysm
   brain tumor
   arteriovenous malformation
   thrombocytopenia, defined as platelet count of <150,000/μl
   known history of coagulopathy
   acute sepsis
   traumatic brain injury (TBI)
   disseminated intravascular coagulation (DIC)
2.2.8. Prior disability due to other disease compromising mRS evaluation, thereby interfering with the primary outcome, operationally defined as an estimated mRS score (by history) of 3 before ICH.
2.2.9. Preexisting unstable epilepsy.
2.2.10. Patients with active systemic bacterial, viral or fungal infections.
2.2.11. Concomitant drug-related exclusion criteria:
   Intravenous immunoglobulin, immunosuppressive and/or chemotherapeutic medications.
   Moderate immunosuppressives (e.g. azathioprine, methotrexate) and/or fingolimod within 2 months prior to randomization.
   Stronger immunosuppressives (e.g. cyclophosphamide, immunosuppressive mAb) within (minimally) 6 months prior to randomization, or longer with long-lasting immunosuppressive medications as determined by the investigator.
2.2.12. Cardiovascular exclusion criteria:
   Cardiac conduction or rhythm disorders including sinus arrest or sino-atrial block, heart rate <50 bpm, sick-sinus syndrome, Mobitz Type II second degree AV block or higher grade AV block, or preexisting atrial fibrillation (either by history or observed at screening).
   PR interval >220 msec. Long QT syndrome or QTcF prolongation >450 msec in males or >470 msec in females on screening electrocardiogram (ECG).
   Patients receiving treatment with QT-prolonging drugs having a long half-life (e.g., amiodarone).
2.2.13. Any of the following abnormal laboratory values prior to randomization:
   White blood cell (WBC) count <2,000/p1 (<2.0×109/L)
   Lymphocyte count <800/p1 (<0.8×109/L)
2.2.14. Pregnant or nursing (lactating) women, where pregnancy is defined as the state of a female after conception and until the termination of gestation, confirmed by a positive hCG laboratory test.
2.2.15. Patients with any other medically unstable condition or serious laboratory abnormality as determined by the investigator.

2.3. Prohibited Treatment

Use of medications displayed in Table 3-1 is not allowed during treatment with siponimod due to increased risk of immunosuppression, confounding of efficacy and/or potential interaction with study treatment (NB: CYP2C9 and CYP3A4 are the major metabolizing enzymes for siponimod).

TABLE 3-1
Prohibited Medications

| Medication | Action to be taken |
|---|---|
| Immunosuppressive/chemo- | Stop taking. If not possible, |
| therapeutic medications or | consider discontinuation |
| procedures, including cyclo- | of study treatment |
| sporine, azathioprine, metho- | |
| trexate, and immunomodulatory | |
| mABs | |
| Medication that suppress AV | Stop taking. If not possible, |
| conduction with the exception | consider discontinuation of |
| of beta-blockers (e.g. carbama- | study treatment |
| zepine, non-dihydropyridine | |
| calcium-channel blockers, or | |
| cardiac glycosides) | |
| Strong inhibitors of CYP2C9 | Stop taking. If not possible, |
| or CYP3A4 | consider discontinuation |
| | of study treatment |
| | Assess ECG and monitor |
| | lymphocyte counts |
| Potent inducers of CYP2C9 | Stop taking. If not possible, |
| | consider discontinuation |
| | of study treatment |

Only potent CYP2C9 and CYP3A4 inhibitors may have a significant effect on siponimod exposure and should not be co-administered with siponimod to avoid or minimize liver events. Potent CYP2C9 and/or CYP3A4 inducers should not be coadministered with siponimod to avoid a potential decrease of efficacy of siponimod in case of under-exposure due to CYP2C9/CYP3A4 induction (note that topical use is permitted).

TABLE 3-2
Typical inhibitors of CYP2C9 or CYP3A4

| Antibiotics: | Antivirals: |
|---|---|
| Clarithromycin | Boceprevir |
| Sulfaphenazole | Telaprevir |
| Telithromycin | Others: |
| Troleandomycin | Amiodarone |
| Protease Inhibitors: | Ataciguat |
| Indinavir | Azapropazone |
| Lopinavir | Benzbromarone |
| Nelfinavir | Bucolome |
| Ritonavir | Cobicistat |
| Saquinavir | Conivaptan |
| Tipranavir | Elvitegravir |
| Antifungals: | Mibefradil |
| Fluconazole | Nefazodone |
| Itraconazole | Oxandrolone |
| Ketoconazole | Tielinic Acid |
| Miconazole | |
| Posaconazole | |
| Voricnazole | |

TABLE 3-3
Typical inducers of CYP2C9 and/or CYP3A4

| Aprepitant | Ginkgo | Rifabutin |
|---|---|---|
| Avasamide | Lersivirine | Rifampin |
| Bosentan | Lopinavir | Ritonavir |
| Carbamazepine | Mitotane | Secobarbital |
| Dalcetrapid | Modafinil | Semagacestat |
| Efavirenz | Nafcillin | St. John's wort |
| Enzalutamide | Nelfinavir | Talviraline |
| Escalicarbazepine | Nevirapine | Thioridazine |
| Etravirin | Phenobarbital | Tipranavir |
| Genistein | Phenytoin | Vigabatrin |

### 3. Study Design

This is a randomized, doubled-blinded, placebo-controlled, parallel group study of siponimod on top of standard-of-care for ICH, consisting of 3 epochs: Screening/Baseline, Treatment, and Follow-Up (see FIG. 1).

3.1. Screening/Baseline Epoch

The screening/baseline epoch lasts no longer than 24 hours from the time of onset of ICH, defined as the time the patient was last witnessed to be at their normal neurological baseline, and consists of:
The initial diagnostic neuroimaging study (CT or MRI) to determine the cause of stroke
Obtaining informed consent
Determining the Glasgow Coma Scale (GCS,) score on presentation
Obtaining medical history, including current medications
Hospital admission laboratory studies
Electrocardiogram (ECG)
Pregnancy test for premenopausal female patients
Vital signs and physical examination, including neurological examination, and
Determination of NIH Stroke Scale (NIHSS) score on presentation

### 3.2. Treatment Epoch

Patients fulfilling all eligibility criteria are randomly allocated to one of two treatment groups in a ratio of 1:1. The treatment start as soon as possible and no later than 24 h after the time of the ICH, defined as the time the patient was last witnessed to be healthy, defined as functioning at their normal, pre-event neurological baseline.

The total treatment lasts 14 days (see FIG. 1):
7 days of i.v. siponimod with titration to the final daily dose of 10 mg/day;
During the 7 days of i.v. infusion treatment, all patients undergo a swallowing safety evaluation per the treating hospital's institutional guidelines and practices.
If the patients pass a swallowing safety evaluation, 7 days of 10 mg siponimod p.o. QD.
Patients who do not successfully pass a swallowing safety evaluation is not transitioned to the p.o. phase of treatment, and siponimod is discontinued after Day 7; but they are not be terminated from the study. These patients continue to be followed for the remainder of the Assessment schedule.

i.v. Dose Titration

The dose titration schedule is based on estimations of the cardiovascular effects of Siponimod balanced with the therapeutic need to achieve fast, effective siponimod concentrations in ICH patients, where the timely achievement of expected therapeutic concentrations may be of great importance.

The siponimod i.v. titration schedule is as follows:
Day 1: 0.25 mg over 6 hours (×2), then 0.5 mg over 6 hours, then 0.75 mg over 6 hours for a total Day 1 dose of 1.75 mg
Day 2: 1.25 mg over 6 hours, then 2 mg over 6 hours, then 2.5 mg over 6 hours (×2) for a total Day 2 dose of 8.25 mg
Days 3 through 7: 2.5 mg over 6 hours (×4) for a total daily dose of 10 mg.

During the i.v. up-titration period patients are closely monitored. Special attention is given to the monitoring of the HR and cardiac rhythm, facilitated by continuous CV telemetry in the Stroke Unit/Intensive Care Unit (ICU) setting. In case of symptomatic bradycardia or cardiac rhythm abnormalities (e.g. atrioventricular blocks or sinus pauses), the Investigator should consider postponing/skipping a dose. Under those predefined conditions a dose may be postponed or skipped, but not more than 2 times in a row. Once patients have completed the 7-day i.v. phase of treatment, they may be discharged to home or transferred to a rehabilitation facility, at the Investigator and/or treating physician's discretion.

During the 7 days of i.v. infusion treatment, all patients undergo a swallowing safety evaluation. Those ICH patients who do not successfully pass a swallowing safety evaluation per the treating hospital's institutional guidelines and practices are not be transitioned to the p.o. phase of treatment, and siponimod is discontinued after Day 7.

Bradycardia with S1P modulators is usually benign, transient, and does not require treatment (Schmouder et al. 2012). The patient is assessed to determine if treatment continuation is acceptable to the treating physician and the Investigator (e.g., 1st or 2nd degree AV blocks) and treatment is continued once the patient recovers from symptomatic bradycardia. In the case of 3rd degree AV block and/or a hemodynamically-affected patient, the treatment is not reinitiated.

Any reduction in heart rate, which, in the opinion of the Investigator or treating intensivist, is clinically significant and requires intervention (e.g., acutely altered mental status, ongoing severe ischemic chest pain, congestive heart failure, hypotension, or other signs of shock) is treated according to standard medical practice, and suggested treatment would include: (i) Anticholinergics (e.g. atropine subcutaneous or i.v.) or (ii) Beta-agonists/sympathomimetics (e.g. dopamine or epinephrine). Dosing of these is individualized with respect to the desired clinical effect by the treating intensivist.

p.o. Dose

Eligible patients who pass a swallowing safety evaluation continue with 7-day p.o. phase of treatment with siponimod 10 mg QD (daily dose). During the Treatment Epoch, all patients undergo study-specific assessments according to the Assessment Schedule (Table 4).

TABLE 4
Assessment schedule

| | Study Phase | | | | | | | | | | | | |
|---|---|---|---|---|---|---|---|---|---|---|---|---|---|
| | Screening/ | | | | | | | | | | | | |
| | Baseline | Treatment | | | | | | | | | Follow-up | | |
| | Visit Numbers¹ | | | | | | | | | | | | |
| | V1 | V2 | V3 | V4 | V5 | V6 | V7 | V8 | V9 | V10 | V11 | V12 | V13 |
| | Days | | | | | | | | | | | | |
| | −1 | 1 | 2 | 3 | 4 | 5 | 6 | 7 | 8 | 14 | 21 | 30 ± 1 | 90 ± 2 |
| Informed consent | X | | | | | | | | | | | | |
| Glasgow Coma Scale | X | | | | | | | | | | | | |
| Medical history/current | X | | | | | | | | | X | X | X | X* |
| medical conditions | | | | | | | | | | | | | |
| Routine Clinical Laboratory | X | X | X | X | | | | X | | X | X | X | X* |
| Tests | | | | | | | | | | | | | |
| ECG evaluation | X | X | X | X | | | | X | | | | | |
| Pregnancy and | X | | | | | | | | | | | X | X* |
| assessments of fertility | | | | | | | | | | | | | |
| Inclusion/Exclusion criteria | X | | | | | | | | | | | | |
| Vital Signs | X | X | X | X | X | X | X | X | X | X | X | X | X* |
| NIHSS | X | X | X | X | X | X | X | X | | | | | X* |
| Physical examination | X | | | | | | | | | X | | X | X* |
| Neurological Examination | X | | | | | | | | | X | | X | X* |
| CYP2C9 Genotyping | | X | | | | | | | | | | | |
| Dose-i.v. infusion | | X | X | X | X | X | X | X | | | | | |
| Dose-p.o. QD | | | | | | | | | X | X | | | |
| PK blood collection | | X² | | | | | | | X³ | X³ | | | |
| CT scan | SoC⁴ | SoC⁴ | | | | | | X | | X | | | |
| Modified Rankin Scale | | | | | | | | | | X | | X | X* |
| (mRS) | | | | | | | | | | | | | |
| Actigraphy | | | | | | | | | | X | | X | X |
| C-SSRS | | | | | | | | | | X | X | X | X |
| Exploratory Serum | | X | | | | | | X | | X | | | X |
| Biomarkers | | | | | | | | | | | | | |
| Exploratory Plasma | | X | | | | | | X | | X | | | X |
| Biomarkers | | | | | | | | | | | | | |
| Pharmacogenetic Informed | | X | | | | | | | | | | | |
| Consent | | | | | | | | | | | | | |
| Exploratory DNA Sampling | | X | | | | | | | | | | | |
| (optional) | | | | | | | | | | | | | |
| Concomitant therapies | X | X | X | X | X | X | X | X | X | X | X | X | X |
| Adverse events | X | X | X | X | X | X | X | X | X | X | X | X | X* |
| Serious adverse events | X | X | X | X | X | X | X | X | X | X | X | X | X* |
| Study completion | | | | | | | | | | | | | X* |
| information | | | | | | | | | | | | | |

¹Visit structure given for internal programming purpose only
²PK samples at 0.5 hr, 2 hr, and 6 hr after start of first infusion; 2 mL at each time point
³pre oral dose
⁴Standard of Care
*Assessments for discontinued patients.

Follow-Up Epoch

Patients return for scheduled outpatient (or inpatient, if still in rehabilitation facility) follow-up visits after being discharged from the ICU or inpatient hospital floor, according to the Assessment schedule. The Follow-Up Epoch lasts until Day 90 after ICH.

### 4. Study Treatment

4.1. Investigational treatment and control drug(s)

TABLE 5
Overview of study medication

| Study drug | | Appear- | | Pack- |
|---|---|---|---|---|
| name | Formulation | ance | Unit dose | aging |
| Siponimod/ | Film-coated | White | 2 mg/0 mg | Double |
| placebo | Tablet | round | | blind |
| | | tablet | | kits |
| Siponimod/ | Concentrate for | 6 ml | 3.5 mg/ | Double |
| placebo | Solution for | vial | 3.5 mL | blind |
| | Infusion | | for siponimod; | kits |
| | | | 0 mg/3.5 mL | |
| | | | for placebo | |

4.2. Additional study treatment

All patients receive standard of treatment and care for patients with ICH according to the AHA/ASA (Hemphill et al. 2015) and ESO Guidelines (Steiner et al. 2014). No additional treatment beyond investigational treatment is required for this trial. General Stroke Unit/Intensive Care Unit management throughout the study needs to be recorded on the Concomitant Medication eCRF. Post ICH rehabilitation, dates and therapy sessions should also be recorded on the same CRF.

4.3. Treatment arms

Patients are assigned to one of the following 2 treatment arms in a ratio of 1:1.
Study treatments are defined as:
Siponimod
   Day 1: i.v. 0.25 mg over 6 hours (×2), then 0.5 mg over 6 hours, then 0.75 mg over 6 hours for a total Day 1 dose of 1.75 mg
   Day 2: i.v. 1.25 mg over 6 hours, then 2 mg over 6 hours, then 2.5 mg over 6 hours (×2) for a total Day 2 dose of 8.25 mg
   Days 3 through 7: i.v. 2.5 mg over 6 hours (×4) for a total daily dose of 10 mg
   Days 8 through 14; 10 mg p.o. QD

or
Placebo
   Days 1 through 7: matching I.V. placebo
   Days 8 through 14; matching p.o. placebo

### 5. Efficacy/Pharmacodynamics

### 5.1 Clinical Outcome Assessments (COAs)

5.1.1 Modified Rankin Scale (mRS)

The modified Rankin Scale (mRS), is a widely-used, clinician-assessed instrument, and is considered the current standard assessment for stroke outcomes by most Health Authorities. It consists of 6 grades of disability, higher scores indicating more severe disability (0=asymptomatic, 6=dead).

| | |
|---|---|
| 0 | No symptoms |
| 1 | No significant disability. Able to carry out all usual activities, despite |
| 2 | Slight disability. Able to look after own affairs without assistance, |
| | but unable to carry out all previous activities |
| 3 | Moderate disability. Requires some help, but able to walk unassisted |
| 4 | Moderately severe disability. Unable to attend to own bodily needs |
| | without assistance, and unable to walk unassisted |
| 5 | Severe disability. Requires constant nursing care and attention, |
| | bedridden, incontinent |
| 6 | Dead |

(Stroke. 2017; 48-2017 American Heart Association, Inc.-Joseph P. Broderick, et al.)

The strength of the mRS is that it captures the full spectrum of limitations in activity and participation after stroke. The inter-rater reliability of the scale is moderate and improves significantly with structured interviews (0.56 versus 0.78; Banks and Marotta 2007); and this structured approach is used in our study (Wilson et al 2002, Wilson et al 2005). The mRS can be administered by investigators, study nurses, and research assistants. Training in administration of the structured mRS interview is provided to site personnel as necessary, and proficiency certification is monitored and centrally recorded. In this study, structured mRS interviews is video recorded, then securely transferred to and rated by a Central Independent Adjudication Panel. Individual (rater) mRS scores (and the panel average) as well as the panel consensus score for each interview is recorded.

The mRS score at 90 days after ICH is the primary endpoint for measuring Siponimod efficacy in this study.

The 90-day mRS score has been used as an endpoint in many stroke studies, including the INTERACT2 (Anderson et al 2013), ATACH (Qureshi et al 2010), SAMURAI-ICH (Koga et al 2014), and ENOS (ENOS Trial Investigators 2015) trials. In addition, a recent study (Murthy et al 2015) using the mRS found an association between early PHE expansion after ICH and poorer 90 day functional outcomes in basal ganglia hemorrhages <30 cc, which is the population chosen for the clinical trial of the present disclosure.

5.1.2 NIH Stroke Scale (NIHSS)

The National Institutes of Health Stroke Scale (NIHSS) is the most widely used clinical instrument to assess the neurological impact of acute stroke (Lyden 2017). The NIHSS consists of 13 individually scored items, with a maximal composite score of 42, higher scores indicating greater stroke severity. The NIHSS is administered by investigators or study nurses. NIHSS training certification is monitored and centrally recorded.

Patients with ICH often experience early neurological deterioration (END) within the first few days after stroke due either to expansion of the initial hematoma or increase in PHE.

Both increased total peripheral white blood cell counts (Sun et al 2012) and greater absolute PHE volume (Rodriguez-Luna et al 2016) have been shown to be associated with END. Various studies and centers define END criteria using different rating scales (GCS, NIHSS) and as occurring within different time windows (24 hours to 7 days) after ICH. For the study of the present disclosure, END is defined as NIHSS worsening by 4 or more points between initial presentation and Day 7 after ICH.

### 6. Safety

6.1 Electrocardiogram (ECG)

Continuous cardiac monitoring is implemented via bedside monitoring in all patients during days when the patient is in the stroke/intensive care unit. Cardiac monitoring is performed from 1 hour before dosing and up to 48 hours after the first drug administration. Continuous cardiac monitoring is done for a longer duration on a case-by-case basis, depending on the patient's conditions. Standard twelve-lead ECGs is performed for all patients at the time points as indicated in Table 4.

Cardiac safety monitoring data is used for cardiac rhythm evaluation (mainly bradyarrhythmias, such as atrioventricular blocks and sinus pauses: Frequency and duration of sinus pauses (>2 seconds)) and for heart rate (HR) assessments.

7. Other assessments

7.1 CYP2C9 Genotyping

Genotyping is performed to determine whether CYP2C9 genotype influences siponimod pharmacokinetics.

7.2 CT Scan

Studies of the trajectory of perihematomal edema (PHE) assessed with either magnetic resonance (MR) (Venkatasubramanian et al. 2011) or computed tomography (CT) imaging (Staykov et al. 2011) largely agree on the average time course of its development, which increases to a plateau between days 7-14 after ICH. Analyzing data from the VISTA-ICH archive, Murthy et al (2015) showed an association between early PHE expansion within 72 hours after ICH and poorer 90 day functional outcomes in basal ganglia hemorrhages 30 cc in volume.

In this study, following the initial diagnostic CT, repeated CT images is obtained between 24-48 hours after the diagnostic scan (i.e., standard-of-care in ICH), and on Days 7 and 14 after ICH to capture the trajectory of PHE increase and plateau after ICH. Only noncontrast CT scans are obtained on Days 7 and 14. In the analysis it is used the noncontrast scan acquired on each patient at first follow-up (24-48 hours after the diagnostic scan). However it does not dictate the standard-of-care scan protocol for the initial and first follow-up scan beyond ensuring a noncontrast scan is acquired, as it usually happen according to standard practice.

7.3 Actiqraphy

The use of wearable or externally-monitored actigraphy in a variety of neurological and musculoskeletal disorders, including stroke rehabilitation, is growing; and wearable devices, which may or may not provide direct patient feedback, are increasingly used to measure functional mobility and rehabilitation outcomes (Wang et al 2017). The actigraphy devices are similar to a wrist-watch and are lightweight, water-resistant, and can be worn continuously for several days. To measure functional mobility with greater sensitivity, and in a more naturalistic (e.g., home) setting, patients are fitted with wrist-worn actigraphy devices around Days 14, 30, and 90 after ICH.

### 8. Results

The above siponimod dosing regimen, i.e., a two week treatment with siponimod administered daily (7 days i.v. with titration followed by 7 days p.o.) compared to placebo improves global functioning measured by the modified Rankin Scale (mRS) score on Day 90 after ICH, and/or decreases the development of perihematomal edema (PHE) between 24 hours and 14 days after initiation of siponimod treatment; and/or improves functional mobility of the affected body parts.

1. A method of treating stroke in a human subject suffering from stroke said method comprising
(a) intravenously administering to said subject multiple consecutive doses of siponimod over a time period equal to or up to 96 hours calculated starting at the first intravenously administered dose, wherein
(i) the first administered dose is not less than 0.25 mg and not more than 1.25 mg;
and wherein
(ii) each dose of the one or more consecutive doses administered after the first dose is not less than the directly preceding administered dose and not more than the directly subsequent administered dose;
and wherein
(iii) the total sum of the consecutive doses administered over a time period of 24 consecutive hours is lower than the maintenance daily dose; and subsequently
(b) administering a maintenance daily dose of siponimod for a maintenance time period of at least 2 days, wherein
(i) the maintenance daily dose is not less than 2 mg and not more than 20 mg of siponimod.

2. A method of treating stroke in a human subject suffering from stroke according to claim 1, wherein the first administered dose of step (a) is 0.25 mg.

3. A method of treating stroke in a human subject suffering from stroke according to claim 1, wherein the maintenance daily dose of step (b) is 10 mg of siponimod.

4. A method of treating stroke in a human subject suffering from stroke as defined in claim 1, wherein if a consecutive dose in step (a) is increased by an increment, said increment is governed by a modified Fibonacci series, i.e. a given dose is the sum of two directly previous doses ±40%, for example ±35%, for example ±30%, for example ±20%, e.g. about ±23%, or for example ±10%.

5. A method of treating stroke in a human subject suffering from stroke according to claim 1, wherein the maintenance daily dose of siponimod administered in step (b) is administered for a maintenance time period of at least 3 days, e.g. 5 days.

6. A method of treating stroke in a human subject suffering from stroke according to claim 1, wherein the administration of the maintenance daily dose of siponimod in step (b) is carried out in a first phase by intravenous administration and in a second phase by oral administration.

7. A method of treating stroke in a human subject suffering from stroke, according to claim 1, comprising
(a) intravenously administering to said subject multiple consecutive doses of siponimod over 48 hours calculated starting at the first intravenously administered dose, wherein
on Day 1 the administered doses are 0.25 mg over 6 hours, then 0.25 mg over 6 hours, then 0.5 mg over 6 hours, and then 0.75 mg over 6 hours for a total Day 1 dose of 1.75 mg; and
on Day 2 the administered doses are 1.25 mg over 6 hours, then 2 mg over 6 hours, then 2.5 mg over 6, and then 2.5 mg over 6 hours for a total Day 2 dose of 8.25 mg; and
(b) intravenously administering a daily maintenance dose of 10 mg of siponimod on Day 3 through Day 7; and optionally orally administering a daily maintenance dose of 10 mg of siponimod on and after Day 8, preferably on Day 8 through Day 14; and
(c) optionally continuously monitoring the human subject in need thereof via cardiovascular telemetry for at least the first 24 hours, preferably for at least the first 48 hours calculated starting from the administration of the first dose of siponimod.

8. A method of treating stroke in a human subject suffering from stroke according to claim 1, wherein the administered i.v. composition containing siponimod is directly obtained by diluting a concentrate containing siponimod, wherein said concentrate
(i) is in the form of a liquid;
(ii) contains 1 mg/mL of siponimod; and
(iii) contains 7 wt. %-13 wt. % of 2-hydroxypropyl-f3-cyclodextrin (HPBCD), a buffer agent; and optionally a tonicity agent.

9. A method of treating stroke in a human subject suffering from stroke according to claim 1, wherein when siponimod is administered orally, siponimod is in the form of a co-crystal with fumaric acid.

10. A method of treating a human subject suffering from stroke according to claim 1, wherein stroke is intracerebral hemorrhage (ICH).

11. A method of improving global functioning of a human subject suffering from stroke, preferably from ICH, wherein said method comprises:
(a) intravenously administering to said subject multiple consecutive doses of siponimod over a time period equal to or up to 96 hours calculated starting at the first intravenously administered dose, wherein
(i) the first administered dose is not less than 0.25 mg and not more than 1.25 mg;
and wherein
(ii) each dose of the one or more consecutive doses administered after the first dose is not less than the directly preceding administered dose and not more than the directly subsequent administered dose; and wherein
(iii) the total sum of the consecutive doses administered over a time period of 24 consecutive hours is lower than the maintenance daily dose; and subsequently
(b) administering the maintenance daily dose of siponimod for a maintenance time period of at least 2 days, wherein the maintenance daily dose is not less than 2 mg and not more than 20 mg of siponimod.
